(19) United States
(12) Patent Application Publication
(10) Pub. No.: US 2024/0407996 A1
(43) Pub. Date: Dec. 12, 2024

(54) SUNSCREEN COMPOSITIONS AND METHODS OF USE

(71) Applicant: MARY KAY INC., Addison, TX (US)

(72) Inventors: Wanli ZHAO, Addison, TX (US); Douglas ZUBER, Addison, TX (US)

(73) Assignee: MARY KAY INC., Addison, TX (US)

(21) Appl. No.: 18/680,001

(22) Filed: May 31, 2024

Related U.S. Application Data
(60) Provisional application No. 63/471,383, filed on Jun. 6, 2023.

(51) Int. Cl. A61K 8/73; A61Q 17/04

(52) U.S. Cl. CPC A61K 8/732; A61Q 17/04; A61K 2800/30

(57) ABSTRACT

Disclosed are sunscreen compositions comprising avobenzone, octocrylene, octisalate, and aluminum starch octenylsuccinate. Also disclosed is a method for protecting skin from ultraviolet radiation, the method comprising topically applying to the skin a composition comprising an effective amount of avobenzone, octocrylene, octisalate, and aluminum starch octenylsuccinate.

### CROSS-REFERENCE TO RELATED APPLICATIONS

This application claims the benefit of priority to U.S. Provisional Patent Application Ser. No. 63/471,383, filed Jun. 6, 2023, hereby incorporated by reference in its entirety.

### BACKGROUND

### I. Field of the Invention

The present invention relates generally to sunscreen compositions having a sun protection factor (SPF), and methods including such compositions for protecting skin from the deleterious effects of ultraviolet A (UVA) and ultraviolet B (UVB) radiation exposure from the sun.

### II. Description of Related Art

Certain sunscreen compositions are known in the art. Many traditional sunscreen compositions rely upon chemical ultraviolet (UV) radiation-filtering ingredients to elicit a suitable SPF for protecting skin from UV radiation exposure. These traditional sunscreen compositions produce a greasy, inelegant feel when applied to skin, due to the high concentration of chemical UV filters in the composition necessary to achieve a desired SPF value.

One of the ingredients utilized as a UV filter and as a solubilizing agent for other chemical UV filters in sunscreen compositions is homosalate. Given the safety concerns regarding potential endocrine disrupting properties of homosalate, homosalate is not recommended for use in sunscreen compositions at concentrations of greater 10%. To reduce homosalate in sunscreen compositions but still maintain the desired SPF upon reduction or removal of the homosalate, oils can be added to the compositions. These oils added to replace the homosalate can increase the oily feel of the compositions when applied to skin.

For UV-filtering ingredients to protect the skin evenly (e.g., to avoid variation in levels of UV damage or UV exposure), they must form a uniform layer of composition film covering the skin. Greasy or oily compositions do not spread uniformly on the skin and have a tendency to migrate and pool from point of application or a tendency to be easily removed from the skin. In addition to lacking a pharmaceutically or cosmetically elegant feel, sunscreen compositions having little to no homosalate may also exhibit a reduction in the effective SPF provided by the active UV-filtering ingredients.

Others have attempted to create compositions and methods for protecting skin from UV radiation exposure from the sun when using reduced amounts of or no homosalate. However, many attempts have been ineffective, only addressed one or a few of the undesired outcomes, or have unacceptable side effects. Thus, there is a need for new pharmaceutically or cosmetically elegant sunscreen compositions that include little to no homosalate but remain effective at protecting skin from UV radiation.

### SUMMARY

A discovery has been made that provides a solution to at least one or more of the problems associated with traditional sunscreen compositions. In one aspect, a solution resides in sunscreen compositions having a combination of UVA and UVB filters and aluminum starch octenylsuccinate that provide a desirable SPF (e.g., greater than 30), that protect skin from UVA and UVB radiation, and that have a pharmaceutically or cosmetically elegant feel. In some aspects, such sunscreen compositions include little to no homosalate, yet still exhibit these desirable characteristics. Without wishing to be bound by theory, it is believed that the inclusion of aluminum starch octenylsuccinate in the composition: reduces oiliness or greasiness of the compositions; reduces the greasy, inelegant feel of the compositions when applied to skin; and helps to leave the skin surface less oily. The compositions including aluminum starch octenylsuccinate can also be more easily applied in a uniform layer on the skin, which can increase the effective SPF value of the compositions.

In one aspect, disclosed is a sunscreen composition comprising a combination of organic UV filters and aluminum starch octenylsuccinate. In some aspects, the composition does not include homosalate. In some aspects, the sunscreen compositions may have a sun protection factor (SPF) of at least, at most, exactly, or between (inclusive or exclusive) any two of 20, 25, 30, 35, 40, 45, 50, 55, 60, 65, or 70, including any range or value derivable therein. In some aspects, the sunscreen compositions may have an SPF of about 30. In some aspects, the sunscreen compositions may have an SPF of about 40. In some aspects, the sunscreen compositions may have an SPF of about 50.

In some aspects, the organic UV filters include a combination of avobenzone, octocrylene, and octisalate. In some aspects, the sunscreen compositions comprise 10 to 20% by weight of the organic UV filters. In some aspects, the sunscreen compositions comprise 2% to 5% by weight of avobenzone; 8% to 11% by weight of octocrylene; and/or 2% to 5% by weight, of octisalate. In some aspects, the sunscreen compositions comprise about 3% by weight of avobenzone; about 9% by weight of octocrylene; and/or about 3% by weight of octisalate. In some aspects, the composition includes one or more additional organic UV filters. In some aspects, the composition does not include a water-soluble UV filter.

In some aspects, the sunscreen compositions comprise 0.01% to 5% by weight of aluminum starch octenylsuccinate. In some aspects, the sunscreen compositions comprise about 1% by weight of aluminum starch octenylsuccinate.

Accordingly, in some aspects, the sunscreen compositions of the present disclosure include 2% to 5% by weight of avobenzone; 8% to 11% by weight of octocrylene; 2% to 5% by weight of octisalate; and/or 0.01% to 5% by weight of aluminum starch octenylsuccinate. In some aspects, the sunscreen compositions of the present disclosure include about 3% by weight of avobenzone; about 9% by weight of octocrylene; about 3% by weight of octisalate; and/or about 1% by weight of aluminum starch octenylsuccinate.

In some aspects, the sunscreen compositions may further include one or more ingredients described herein. For example, the sunscreen compositions may further include water, antioxidants, emulsifying agents, surfactants, film-forming agents, chelating agents, moisturizing agents, preservatives, and/or thickening agents.

In some aspects, the sunscreen composition further includes any one of, any combination of, or all of capryl methicone, ceteareth-25, disodium ethylene dicocamide PEG-15 disulfate, PVP/eicosene copolymer, glyceryl stearate, beeswax, glycerin, disodium EDTA, hydroxyacetophenone, propanediol, xanthan gum, ammonium acryloyldimethyltaurate/VP copolymer, phenoxyethanol, ethylhexylglycerin, dimethicone, and/or silica. In some aspects, the sunscreen composition can further include any one of, any combination of, or all of niacinamide, encapsulated resveratrol, oligopeptide-1, and/or Opuntia ficus-indica fruit extract. In some aspects, the sunscreen composition can further include any one of, any combination of, or all of cetearyl alcohol, potassium hydroxide, caprylyl glycol, hexylene glycol, 1,2-hexanediol, decylene glycol, Cestrum latifolium leaf extract, calcium ketogluconate, tripeptide-1, Centella asiatica meristem extract, Silybum marianum extract, 4-t-butylcyclohexanol, sodium PCA, Alpinia galanga leaf extract, and/or Saussurea involucrate extract.

Accordingly, in some aspects, the sunscreen compositions of the present disclosure include avobenzone, octocrylene, octisalate, aluminum starch octenylsuccinate, capryl methicone, ceteareth-25, disodium ethylene dicocamide PEG-15 disulfate, PVP/eicosene copolymer, glyceryl stearate, beeswax, glycerin, disodium EDTA, hydroxyacetophenone, propanediol, xanthan gum, ammonium acryloyldimethyltaurate/VP copolymer, phenoxyethanol, ethylhexylglycerin, dimethicone, and silica. In some aspects, the sunscreen compositions of the present disclosure include avobenzone, octocrylene, octisalate, aluminum starch octenylsuccinate, capryl methicone, ceteareth-25, disodium ethylene dicocamide PEG-15 disulfate, PVP/eicosene copolymer, glyceryl stearate, beeswax, glycerin, disodium EDTA, hydroxyacetophenone, propanediol, xanthan gum, ammonium acryloyldimethyltaurate/VP copolymer, phenoxyethanol, ethylhexylglycerin, dimethicone, silica, niacinamide, encapsulated resveratrol, oligopeptide-1, and Opuntia ficus-indica fruit extract. In some aspects, the sunscreen compositions of the present disclosure include avobenzone, octocrylene, octisalate, aluminum starch octenylsuccinate, capryl methicone, ceteareth-25, disodium ethylene dicocamide PEG-15 disulfate, PVP/eicosene copolymer, glyceryl stearate, beeswax, glycerin, disodium EDTA, hydroxyacetophenone, propanediol, xanthan gum, ammonium acryloyldimethyltaurate/VP copolymer, phenoxyethanol, ethylhexylglycerin, dimethicone, silica, cetearyl alcohol, potassium hydroxide, caprylyl glycol, hexylene glycol, 1,2-hexanediol, decylene glycol, Cestrum latifolium leaf extract, calcium ketogluconate, tripeptide-1, Centella asiatica meristem extract, Silybum marianum extract, 4-t-butylcyclohexanol, sodium PCA, Alpinia galanga leaf extract, and Saussurea involucrate extract.

In some aspects, the sunscreen composition is an emulsion, a lotion, a cream, a gel, a spray, or an ointment. In some aspects, oiliness of the skin surface is not significantly increased after application of the sunscreen composition. In some aspects, the sunscreen composition forms a uniform layer after application to the skin.

In another aspect of the present disclosure, methods of use for the sunscreen compositions comprising a combination of organic UV filters and aluminum starch octenylsuccinate disclosed herein are described. In some aspects, disclosed is a method for protecting skin from ultraviolet radiation. In some aspects, the method includes topically applying to the skin any one of the sunscreen compositions disclosed herein comprising an effective amount of avobenzone, octocrylene, octisalate, and aluminum starch octenylsuccinate. In some aspects, the composition does not include homosalate. In some aspects, the composition does not include a water-soluble UV filter.

The composition can be applied to facial skin, arm skin, hand skin, back skin, neck skin, leg skin, foot skin, stomach area skin, etc. In some aspects, the sunscreen composition is applied to facial skin. In some aspects, the sunscreen composition is applied to skin on a user's arms or hands. In some aspects, the sunscreen composition is combined with one or more other skin care compositions prior to application to the skin.

In some aspects, the skin is prone to sunburn and/or melanin overproduction after sun exposure before application of the composition. In some aspects, the skin is not sunburned. In some aspects, the skin is treated to reduce or prevent sunburn. In some aspects, the skin is treated to reduce or prevent melanin overproduction after sun exposure. In some aspects, oiliness of the skin surface is not significantly increased after application of the composition. In some aspects, the composition forms a uniform layer on the skin surface after application of the composition. In some aspects, the composition is combined with one or more other skin care compositions prior to application to the skin.

In some aspects, the sunscreen compositions of the present disclosure can be pharmaceutically or cosmetically elegant or can have pleasant tactile properties. “Pharmaceutically elegant,” “cosmetically elegant,” and/or “pleasant tactile properties” describes a composition that has particular tactile properties that feel pleasant on the skin (e.g., compositions that are not too watery or greasy or oily, compositions that have a silky texture, compositions that are non-tacky or sticky, etc.). Pharmaceutically or cosmetically elegant can also relate to the creaminess or lubricity properties of the composition or to the moisture retaining properties of the composition.

In some aspects, the sunscreen compositions are applied to skin multiple times per day or per week. In some instances, the composition can be applied to skin 1, 2, 3, 4, 5, or more times per day. In some instances, the composition can be applied to skin 1, 2, 3, 4, 5, 6, 7, or more per week. In some instances, the composition can be applied to skin about 3 times per day. In some instances, the composition can be applied to skin more than 3 times per day. In some instances, the composition can be applied to skin about 7 times per week.

In some aspects, the sunscreen composition can be combined with one or more skin care compositions for treating skin. In some instances, the one or more skin care compositions affect a smoothing, hydrating, and/or age fighting effect on skin. In some instances, the one or more skin care compositions are applied to the skin before application of the sunscreen composition to the skin. In some instances, the one or more skin care compositions are applied to the skin after application of the sunscreen composition to the skin. In some instances, the one or more skin care compositions are applied to the skin at the same time as application of the sunscreen composition to the skin.

In some aspects, the compositions of the present invention are formulated as a topical skin composition. The composition can have a dermatologically acceptable vehicle or carrier for the compounds, compositions and extracts. The compositions of the present invention can also include any one of, any combination of, or all of the following additional ingredients: water, a conditioning agent, a chelating agent, a moisturizing agent, a humectant, a surfactant, an oil, a pH adjuster, inorganic salts, a preservative, a thickening agent, a silicone-containing compound, an essential oil, a structuring agent, a vitamin, a pharmaceutical ingredient, or an antioxidant, other ingredients identified in this specification or those known in the art, or any combination of such ingredients or mixtures of such ingredients. In certain aspects, the composition can include at least two, three, four, five, six, seven, eight, nine, ten, eleven, twelve, thirteen, fourteen, fifteen, sixteen, seventeen, or more, or all of the additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

The compositions, in non-limiting aspects, can have a pH of about 6 to about 9. In some aspects, the pH can be 1, 2, 3, 4, 5, 6, 7, 8, 9, 10, 11, 12, 13, or 14 (including any range or value derivable therein). The compositions can include a triglyceride. Non-limiting examples include small, medium, and large chain triglycerides. In certain aspects, the triglyceride is a medium chain triglyceride (e.g., caprylic capric triglyceride). The compositions can also include preservatives. Non-limiting examples of preservatives include phenoxyethanol, methylparaben, propylparaben, or any mixture of thereof. In some embodiments, the composition is paraben-free.

The composition can be a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solution (e.g., aqueous or hydro-alcoholic solution), anhydrous base (e.g., lipstick or a powder), ointment, milk, paste, aerosol, solid forms, eye jelly, etc. The composition can be in powdered form (e.g., dried, lyophilized, particulate, etc.). The composition can be formulated for topical skin application at least 1, 2, 3, 4, 5, 6, 7, or more times a day during use. In some aspects of the present invention, compositions can be storage stable or color stable, or both. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

In some aspects, the composition is applied to clean skin. It is also contemplated that the compositions disclosed throughout this specification can be used as a leave-on or rinse-off composition. By way of example, a leave-on composition can be one that is topically applied to skin and remains on the skin for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). In some instances, the composition is left on the skin to be absorbed. Alternatively, a rinse-off composition can be a product that is intended to be applied to the skin and then removed or rinsed from the skin (e.g., with water) within a period of time such as less than 5, 4, 3, 2, or 1 minute. An example of a rinse off composition can be a skin cleanser, shampoo, conditioner, or soap. An example of a leave-on composition can be a sunscreen, skin moisturizer, mask, overnight cream, or a day cream.

Kits that include the compositions of the present invention are also contemplated. In certain embodiments, the composition is comprised in a container. The container can be a bottle, dispenser, or package. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

Also contemplated is a product comprising a composition of the present invention. In non-limiting aspects, the product can be a cosmetic product. The cosmetic product can be one of those described in other sections of this specification or those known to a person of skill in the art. Non-limiting examples of products include a sunscreen, a moisturizer, a cream, a lotion, an ointment, a skin softener, a serum, a spray, a gel, a wash, a body butter, a scrub, a foundation, a night cream, a lipstick, a cleanser, a toner, a mask, an anti-aging product, a deodorant, an antiperspirant, a perfume, a cologne, etc.

“Topical application” means to apply or spread a composition onto the surface of lips or keratinous tissue. “Topical skin composition” includes compositions suitable for topical application on skin, lips, and/or keratinous tissue. Such compositions are typically dermatologically acceptable in that they do not have undue toxicity, incompatibility, instability, allergic response, and the like, when applied to lips, skin, and/or keratinous tissue. Topical skin care compositions of the present invention can have a selected viscosity to avoid significant dripping or pooling after application to lips, skin, and/or keratinous tissue.

Also disclosed in the context of the present invention are aspects 1 to 26. Aspect 1 is a sunscreen composition comprising avobenzone, octocrylene, octisalate, and aluminum starch octenylsuccinate. Aspect 2 is the sunscreen composition of aspect 1, wherein the composition does not include homosalate. Aspect 3 is the sunscreen composition of aspect 1 or 2, wherein the composition includes one or more additional organic UV filters. Aspect 4 is the sunscreen composition of any of aspects 1-3, wherein the composition does not include a water-soluble UV filter. Aspect 5 is the sunscreen composition of any of aspects 1-4, wherein the composition comprises: 2% to 5% by weight of the avobenzone; 8% to 11% by weight of the octocrylene; 2% to 5% by weight of the octisalate; and/or 0.01% to 5%, by weight of the aluminum starch octenylsuccinate. Aspect 6 is the sunscreen composition of any of aspects 1-5, wherein the composition comprises: about 3% by weight of the avobenzone; about 9% by weight of the octocrylene; about 3% by weight of the octisalate; and/or about 1% by weight of the aluminum starch octenylsuccinate. Aspect 7 is the sunscreen composition of any one of aspects 1-6, further comprising: water; an antioxidant; an emulsifying agent; a surfactant; a film-forming agent; a chelating agent; a moisturizing agent; a preservative; and/or a thickening agent. Aspect 8 is the sunscreen composition of any one of aspects 1-7, further comprising capryl methicone, ceteareth-25, disodium ethylene dicocamide PEG-15 disulfate (Surfactant), PVP/eicosene copolymer, glyceryl stearate, beeswax, glycerin, disodium EDTA, hydroxyacetophenone, propanediol, xanthan gum, ammonium acryloyldimethyltaurate/VP copolymer, phenoxyethanol, ethylhexylglycerin, dimethicone, and/or silica. Aspect 9 is the sunscreen composition of any one of aspects 1-8, further comprising niacinamide, encapsulated resveratrol, oligopeptide-1, and/or Opuntia ficus-indica fruit extract. Aspect 10 is the sunscreen composition of any one of aspects 1-9, further comprising cetearyl alcohol, potassium hydroxide, caprylyl glycol, hexylene glycol, 1,2-hexanediol, decylene glycol, Cestrum latifolium leaf extract, calcium ketogluconate, tripeptide-1, Centella asiatica meristem extract, Silybum marianum extract, 4-t-butylcyclohexanol, sodium PCA, Alpinia galanga leaf extract, and/or Saussurea involucrate extract. Aspect 11 is the sunscreen composition of any one of aspects 1-10, wherein the composition is an emulsion, a lotion, a gel, or an ointment. Aspect 12 is the sunscreen composition of any one of aspects 1-11, wherein the composition has a sun protection factor (SPF) of at least 20, such as 20 to 50. Aspect 13 is the sunscreen composition of any one of aspects 1-12, wherein the composition has a sun protection factor (SPF) of about 30.

Aspect 14 is a method for protecting skin from ultraviolet radiation, the method comprising topically applying to the skin a composition comprising an effective amount of avobenzone, octocrylene, octisalate, and aluminum starch octenylsuccinate. Aspect 15 is the method of aspect 14, wherein the composition does not include homosalate. Aspect 16 is the method of aspect 14 or 15, wherein the composition does not include a water-soluble UV filter. Aspect 17 is the method of any one of aspects 14-16, wherein the composition comprises: 2% to 5% by weight of the avobenzone; 8% to 11% by weight of the octocrylene; 2% to 5% by weight of the octisalate; and/or 0.1% to 3% by weight of the aluminum starch octenylsuccinate. Aspect 18 is the method of any one of aspects 14-17, wherein the skin is prone to sunburn and/or melanin overproduction after sun exposure and/or wherein the risk of sunburn and/or melanin overproduction in the skin when exposed to sun is reduced by the application of the composition to the skin before the exposure to the sun. Aspect 19 is the method of any one of aspects 14-18, wherein the skin is not sunburned. Aspect 20 is the method of any one of aspects 14-19, wherein the skin is treated to reduce or prevent melanin production in the skin. Aspect 21 is the method of any one of aspects 14-20, wherein the skin is treated to reduce or prevent sunburn. Aspect 22 is the method of any one of aspects 14-21, wherein oiliness of the skin surface is not significantly increased after application of the composition. Aspect 23 is the method of any one of aspects 1-22, where the composition forms a uniform layer on the skin surface after application of the composition. Aspect 24 is the method of any one of aspects 14-23, wherein the composition is applied to facial skin. Aspect 25 is the method of any one of aspects 14-24, wherein the composition is applied to skin on a user's arms or hands. Aspect 26 is the method of any one of aspects 14-25, wherein the composition is combined with one or more other skin care compositions prior to application to the skin. Aspect 27 is the method of any one of aspects 14-26, wherein the composition the sunscreen composition of any one of aspects 1-13.

“Keratinous tissue” includes keratin-containing layers disposed as the outermost protective covering of mammals and includes, but is not limited to, lips, skin, hair, and nails.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase, such as a measurable increase of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is the ability to protect skin from UV radiation (e.g., sun light).

It is contemplated that any aspect discussed in this disclosure can be implemented with respect to any method or composition of the invention, and vice versa. Furthermore, compositions of the disclosure can be used to achieve methods of the disclosure.

Other objects, features and advantages of the present disclosure will become apparent from the following detailed description. It should be understood, however, that the detailed description and the examples, while indicating specific aspects of the disclosure, are given by way of illustration only. Additionally, it is contemplated that changes and modifications within the spirit and scope of the disclosure will become apparent to those skilled in the art from this detailed description.

### DESCRIPTION OF ILLUSTRATIVE EMBODIMENTS

As noted above, the present invention addresses certain needs by providing a sunscreen composition that provides a desirable SPF (e.g., greater than 30) to protect skin from UVA and UVB radiation from sun and that has a pharmaceutically or cosmetically elegant feel. The sunscreen composition comprises a combination of UVA and UVB filters, aluminum starch octenylsuccinate, and optionally, little to no homosalate. In some aspects, the sunscreen compositions have more pleasant tactile properties (e.g., are less greasy or oily when applied to skin) and leave the skin surface less oily compared to traditional sunscreens. In some aspects, the less greasy or oily sunscreen compositions can be more easily applied in a uniform layer of product film covering the skin, which can increase the effective SPF value of the compositions.

Some compositions of the present disclosure are designed to work as a topical composition. The composition relies on a unique combination of any one of, any combination of, or all of octisalate, avobenzone, octocrylene, and aluminum starch octenylsuccinate. These combinations can be used to create topical compositions that protect skin from ultraviolet radiation, reduce or prevent sunburn, and/or reduce or prevent melanin overproduction caused by sun exposure and/or UV radiation exposure. Non-limiting examples of such compositions are provided in Tables 1-5 of Examples 1-2 below.

Some compositions disclosed herein can be applied to the skin and remain on the skin for a period of time (e.g., at least 1, 2, 3, 4, 5, 10, 20, 30, or 60 minutes or more), after which, the composition, if needed, can be reapplied or rinsed from the skin. Some compositions are designed to be left on skin. Some compositions disclosed herein can be applied to the skin and absorbed at least in part by the skin. Some compositions disclosed herein can be applied to the skin and immediately rinsed from the skin.

These and other non-limiting aspects of the present invention are described in the following sections.

### I. Active Ingredients

Aspects of the present disclosure including sunscreen compositions comprising a combination of combination of UVA and UVB filters and aluminum starch octenylsuccinate that can be topically applied to protect skin from UV radiation (e.g., sun light), reduce or prevent sunburn, and/or reduce or prevent melanin overproduction due to sun exposure and/or UV radiation exposure. The combination of UVA and UVB filters can include at least avobenzone, octocrylene, and octisalate.

Avobenzone, also known as butylmethoxydibenzoylmethane or 4-tert-butyl-4′-methoxydibenzoylmethane, is an oil soluble chemical agent having the structure:

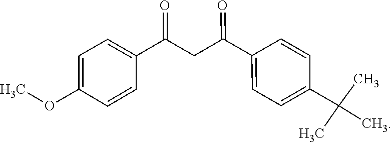 [SMILES: COc1ccc(C(=O)CC(=O)c2ccc(C(C)(C)C)cc2)cc1]

Avobenzone is capable of absorbing UV radiation from approximately 310-400 nm, which covers the UVA range. Avobenzone is one of the very few chemical sunscreens with good coverage of UVA spectrum and is used in products to absorb the full spectrum of UVA rays. Avobenzone degrades in sunlight and loses effectiveness over time. It is typically used with a stabilizer (e.g., octocrylene or homosalate). It is an organic compound and is commercially available from a wide range of sources.

In some aspects, the compositions comprise 2% to 5% by weight of avobenzone. In non-limiting aspects, for example, the compositions can comprise, consist essentially of, or consist of, in their final form, for example, at least, at most, exactly, between (inclusive or exclusive), or about 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%, 3%, 3.01%, 3.02%, 3.03%, 3.04%, 3.05%, 3.06%, 3.07%, 3.08%, 3.09%, 3.1%, 3.11%, 3.12%, 3.13%, 3.14%, 3.15%, 3.16%, 3.17%, 3.18%, 3.19%, 3.2%, 3.21%, 3.22%, 3.23%, 3.24%, 3.25%, 3.26%, 3.27%, 3.28%, 3.29%, 3.3%, 3.31%, 3.32%, 3.33%, 3.34%, 3.35%, 3.36%, 3.37%, 3.38%, 3.39%, 3.4%, 3.41%, 3.42%, 3.43%, 3.44%, 3.45%, 3.46%, 3.47%, 3.48%, 3.49%, 3.5%, 3.51%, 3.52%, 3.53%, 3.54%, 3.55%, 3.56%, 3.57%, 3.58%, 3.59%, 3.6%, 3.61%, 3.62%, 3.63%, 3.64%, 3.65%, 3.66%, 3.67%, 3.68%, 3.69%, 3.7%, 3.71%, 3.72%, 3.73%, 3.74%, 3.75%, 3.76%, 3.77%, 3.78%, 3.79%, 3.8%, 3.81%, 3.82%, 3.83%, 3.84%, 3.85%, 3.86%, 3.87%, 3.88%, 3.89%, 3.9%, 3.91%, 3.92%, 3.93%, 3.94%, 3.95%, 3.96%, 3.97%, 3.98%, 3.99%, 4%, 4.01%, 4.02%, 4.03%, 4.04%, 4.05%, 4.06%, 4.07%, 4.08%, 4.09%, 4.1%, 4.11%, 4.12%, 4.13%, 4.14%, 4.15%, 4.16%, 4.17%, 4.18%, 4.19%, 4.2%, 4.21%, 4.22%, 4.23%, 4.24%, 4.25%, 4.26%, 4.27%, 4.28%, 4.29%, 4.3%, 4.31%, 4.32%, 4.33%, 4.34%, 4.35%, 4.36%, 4.37%, 4.38%, 4.39%, 4.4%, 4.41%, 4.42%, 4.43%, 4.44%, 4.45%, 4.46%, 4.47%, 4.48%, 4.49%, 4.5%, 4.51%, 4.52%, 4.53%, 4.54%, 4.55%, 4.56%, 4.57%, 4.58%, 4.59%, 4.6%, 4.61%, 4.62%, 4.63%, 4.64%, 4.65%, 4.66%, 4.67%, 4.68%, 4.69%, 4.7%, 4.71%, 4.72%, 4.73%, 4.74%, 4.75%, 4.76%, 4.77%, 4.78%, 4.79%, 4.8%, 4.81%, 4.82%, 4.83%, 4.84%, 4.85%, 4.86%, 4.87%, 4.88%, 4.89%, 4.9%, 4.91%, 4.92%, 4.93%, 4.94%, 4.95%, 4.96%, 4.97%, 4.98%, 4.99%, or 5% by weight of avobenzone.

Octocrylene is an oil soluble, water resistant chemical agent comprising an ester formed by the condensation of a diphenylcyanoacrylate with 2-ethylhexanol and has the following structure:

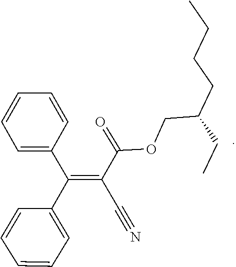 [SMILES: CCCC[C@H](CC)COC(=O)C(C#N)=C(c1ccccc1)c1ccccc1]

Octocrylene is a viscous, oily liquid that is clear and colorless. The extended conjugation of the acrylate portion of the molecule absorbs UV radiation from approximately 280-350 nm, which covers the UVB and short UVA (a.k.a. UVA-2) range, protecting the skin from direct DNA damage. Octocrylene is a stable but relatively weak sunscreen, and is generally inadequate when used alone. It is capable of stabilizing other UV absorbers (e.g., avobenzone). The ethylhexanol portion is a fatty alcohol, adding emollient and oil-like (water resistant) properties. Octocrylene is commercially available from a wide range of sources.

In some aspects, the compositions comprise 8% to 11% by weight of octocrylene. In non-limiting aspects, for example, the compositions can comprise, consist essentially of, or consist of, in their final form, for example, at least, at most, exactly, between (inclusive or exclusive), or about 8%, 8.01%, 8.02%, 8.03%, 8.04%, 8.05%, 8.06%, 8.07%, 8.08%, 8.09%, 8.1%, 8.11%, 8.12%, 8.13%, 8.14%, 8.15%, 8.16%, 8.17%, 8.18%, 8.19%, 8.2%, 8.21%, 8.22%, 8.23%, 8.24%, 8.25%, 8.26%, 8.27%, 8.28%, 8.29%, 8.3%, 8.31%, 8.32%, 8.33%, 8.34%, 8.35%, 8.36%, 8.37%, 8.38%, 8.39%, 8.4%, 8.41%, 8.42%, 8.43%, 8.44%, 8.45%, 8.46%, 8.47%, 8.48%, 8.49%, 8.5%, 8.51%, 8.52%, 8.53%, 8.54%, 8.55%, 8.56%, 8.57%, 8.58%, 8.59%, 8.6%, 8.61%, 8.62%, 8.63%, 8.64%, 8.65%, 8.66%, 8.67%, 8.68%, 8.69%, 8.7%, 8.71%, 8.72%, 8.73%, 8.74%, 8.75%, 8.76%, 8.77%, 8.78%, 8.79%, 8.8%, 8.81%, 8.82%, 8.83%, 8.84%, 8.85%, 8.86%, 8.87%, 8.88%, 8.89%, 8.9%, 8.91%, 8.92%, 8.93%, 8.94%, 8.95%, 8.96%, 8.97%, 8.98%, 8.99%, 9%, 9.01%, 9.02%, 9.03%, 9.04%, 9.05%, 9.06%, 9.07%, 9.08%, 9.09%, 9.1%, 9.11%, 9.12%, 9.13%, 9.14%, 9.15%, 9.16%, 9.17%, 9.18%, 9.19%, 9.2%, 9.21%, 9.22%, 9.23%, 9.24%, 9.25%, 9.26%, 9.27%, 9.28%, 9.29%, 9.3%, 9.31%, 9.32%, 9.33%, 9.34%, 9.35%, 9.36%, 9.37%, 9.38%, 9.39%, 9.4%, 9.41%, 9.42%, 9.43%, 9.44%, 9.45%, 9.46%, 9.47%, 9.48%, 9.49%, 9.5%, 9.51%, 9.52%, 9.53%, 9.54%, 9.55%, 9.56%, 9.57%, 9.58%, 9.59%, 9.6%, 9.61%, 9.62%, 9.63%, 9.64%, 9.65%, 9.66%, 9.67%, 9.68%, 9.69%, 9.7%, 9.71%, 9.72%, 9.73%, 9.74%, 9.75%, 9.76%, 9.77%, 9.78%, 9.79%, 9.8%, 9.81%, 9.82%, 9.83%, 9.84%, 9.85%, 9.86%, 9.87%, 9.88%, 9.89%, 9.9%, 9.91%, 9.92%, 9.93%, 9.94%, 9.95%, 9.96%, 9.97%, 9.98%, 9.99%, 10%, 10.01%, 10.02%, 10.03%, 10.04%, 10.05%, 10.06%, 10.07%, 10.08%, 10.09%, 10.1%, 10.11%, 10.12%, 10.13%, 10.14%, 10.15%, 10.16%, 10.17%, 10.18%, 10.19%, 10.2%, 10.21%, 10.22%, 10.23%, 10.24%, 10.25%, 10.26%, 10.27%, 10.28%, 10.29%, 10.3%, 10.31%, 10.32%, 10.33%, 10.34%, 10.35%, 10.36%, 10.37%, 10.38%, 10.39%, 10.4%, 10.41%, 10.42%, 10.43%, 10.44%, 10.45%, 10.46%, 10.47%, 10.48%, 10.49%, 10.5%, 10.51%, 10.52%, 10.53%, 10.54%, 10.55%, 10.56%, 10.57%, 10.58%, 10.59%, 10.6%, 10.61%, 10.62%, 10.63%, 10.64%, 10.65%, 10.66%, 10.67%, 10.68%, 10.69%, 10.7%, 10.71%, 10.72%, 10.73%, 10.74%, 10.75%, 10.76%, 10.77%, 10.78%, 10.79%, 10.8%, 10.81%, 10.82%, 10.83%, 10.84%, 10.85%, 10.86%, 10.87%, 10.88%, 10.89%, 10.9%, 10.91%, 10.92%, 10.93%, 10.94%, 10.95%, 10.96%, 10.97%, 10.98%, 10.99%, or 11% by weight of octocrylene.

Octisalate, also known as octyl salicylate or 2-ethylhexyl salicylate, is a colorless oily liquid comprising an ester formed by the condensation of salicylic acid with 2-ethylhexanol and has the following structure:

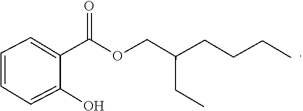 [SMILES: CCCCC(CC)COC(=O)c1ccccc1O]

Octisalate absorbs UVB (ultraviolet) rays from the sun. The salicylate portion of the molecule absorbs ultraviolet light, protecting skin from the harmful effects of exposure to sunlight. The ethylhexanol portion is a fatty alcohol, adding emollient and oil-like (water resistant) properties. It can also stabilize avobenzone and result in longer-lasting sun protection.

In some aspects, the compositions comprise 2% to 5% by weight of octisalate. In non-limiting aspects, for example, the compositions can comprise, consist essentially of, or consist of, in their final form, for example, at least, at most, exactly, between (inclusive or exclusive), or about 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%, 3%, 3.01%, 3.02%, 3.03%, 3.04%, 3.05%, 3.06%, 3.07%, 3.08%, 3.09%, 3.1%, 3.11%, 3.12%, 3.13%, 3.14%, 3.15%, 3.16%, 3.17%, 3.18%, 3.19%, 3.2%, 3.21%, 3.22%, 3.23%, 3.24%, 3.25%, 3.26%, 3.27%, 3.28%, 3.29%, 3.3%, 3.31%, 3.32%, 3.33%, 3.34%, 3.35%, 3.36%, 3.37%, 3.38%, 3.39%, 3.4%, 3.41%, 3.42%, 3.43%, 3.44%, 3.45%, 3.46%, 3.47%, 3.48%, 3.49%, 3.5%, 3.51%, 3.52%, 3.53%, 3.54%, 3.55%, 3.56%, 3.57%, 3.58%, 3.59%, 3.6%, 3.61%, 3.62%, 3.63%, 3.64%, 3.65%, 3.66%, 3.67%, 3.68%, 3.69%, 3.7%, 3.71%, 3.72%, 3.73%, 3.74%, 3.75%, 3.76%, 3.77%, 3.78%, 3.79%, 3.8%, 3.81%, 3.82%, 3.83%, 3.84%, 3.85%, 3.86%, 3.87%, 3.88%, 3.89%, 3.9%, 3.91%, 3.92%, 3.93%, 3.94%, 3.95%, 3.96%, 3.97%, 3.98%, 3.99%, 4%, 4.01%, 4.02%, 4.03%, 4.04%, 4.05%, 4.06%, 4.07%, 4.08%, 4.09%, 4.1%, 4.11%, 4.12%, 4.13%, 4.14%, 4.15%, 4.16%, 4.17%, 4.18%, 4.19%, 4.2%, 4.21%, 4.22%, 4.23%, 4.24%, 4.25%, 4.26%, 4.27%, 4.28%, 4.29%, 4.3%, 4.31%, 4.32%, 4.33%, 4.34%, 4.35%, 4.36%, 4.37%, 4.38%, 4.39%, 4.4%, 4.41%, 4.42%, 4.43%, 4.44%, 4.45%, 4.46%, 4.47%, 4.48%, 4.49%, 4.5%, 4.51%, 4.52%, 4.53%, 4.54%, 4.55%, 4.56%, 4.57%, 4.58%, 4.59%, 4.6%, 4.61%, 4.62%, 4.63%, 4.64%, 4.65%, 4.66%, 4.67%, 4.68%, 4.69%, 4.7%, 4.71%, 4.72%, 4.73%, 4.74%, 4.75%, 4.76%, 4.77%, 4.78%, 4.79%, 4.8%, 4.81%, 4.82%, 4.83%, 4.84%, 4.85%, 4.86%, 4.87%, 4.88%, 4.89%, 4.9%, 4.91%, 4.92%, 4.93%, 4.94%, 4.95%, 4.96%, 4.97%, 4.98%, 4.99%, or 5% by weight of octisalate.

Aluminum starch octenylsuccinate is the aluminum salt of the reaction product of octenylsuccinic anhydride with starch. It is a synthetic, powdery thickening agent, absorbent, viscosity-increasing agent, and anti-caking agent. It can also promote spreadability of products. When included in products, it can provide a powder-like matte finish and fast dry time.

In some aspects, the compositions comprise 0.01% to 5% by weight of aluminum starch octenylsuccinate. In non-limiting aspects, for example, the compositions can comprise, consist essentially of, or consist of, in their final form, for example, at least, at most, exactly, between (inclusive or exclusive), or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.01%, 1.02%, 1.03%, 1.04%, 1.05%, 1.06%, 1.07%, 1.08%, 1.09%, 1.1%, 1.11%, 1.12%, 1.13%, 1.14%, 1.15%, 1.16%, 1.17%, 1.18%, 1.19%, 1.2%, 1.21%, 1.22%, 1.23%, 1.24%, 1.25%, 1.26%, 1.27%, 1.28%, 1.29%, 1.3%, 1.31%, 1.32%, 1.33%, 1.34%, 1.35%, 1.36%, 1.37%, 1.38%, 1.39%, 1.4%, 1.41%, 1.42%, 1.43%, 1.44%, 1.45%, 1.46%, 1.47%, 1.48%, 1.49%, 1.5%, 1.51%, 1.52%, 1.53%, 1.54%, 1.55%, 1.56%, 1.57%, 1.58%, 1.59%, 1.6%, 1.61%, 1.62%, 1.63%, 1.64%, 1.65%, 1.66%, 1.67%, 1.68%, 1.69%, 1.7%, 1.71%, 1.72%, 1.73%, 1.74%, 1.75%, 1.76%, 1.77%, 1.78%, 1.79%, 1.8%, 1.81%, 1.82%, 1.83%, 1.84%, 1.85%, 1.86%, 1.87%, 1.88%, 1.89%, 1.9%, 1.91%, 1.92%, 1.93%, 1.94%, 1.95%, 1.96%, 1.97%, 1.98%, 1.99%, 2%, 2.01%, 2.02%, 2.03%, 2.04%, 2.05%, 2.06%, 2.07%, 2.08%, 2.09%, 2.1%, 2.11%, 2.12%, 2.13%, 2.14%, 2.15%, 2.16%, 2.17%, 2.18%, 2.19%, 2.2%, 2.21%, 2.22%, 2.23%, 2.24%, 2.25%, 2.26%, 2.27%, 2.28%, 2.29%, 2.3%, 2.31%, 2.32%, 2.33%, 2.34%, 2.35%, 2.36%, 2.37%, 2.38%, 2.39%, 2.4%, 2.41%, 2.42%, 2.43%, 2.44%, 2.45%, 2.46%, 2.47%, 2.48%, 2.49%, 2.5%, 2.51%, 2.52%, 2.53%, 2.54%, 2.55%, 2.56%, 2.57%, 2.58%, 2.59%, 2.6%, 2.61%, 2.62%, 2.63%, 2.64%, 2.65%, 2.66%, 2.67%, 2.68%, 2.69%, 2.7%, 2.71%, 2.72%, 2.73%, 2.74%, 2.75%, 2.76%, 2.77%, 2.78%, 2.79%, 2.8%, 2.81%, 2.82%, 2.83%, 2.84%, 2.85%, 2.86%, 2.87%, 2.88%, 2.89%, 2.9%, 2.91%, 2.92%, 2.93%, 2.94%, 2.95%, 2.96%, 2.97%, 2.98%, 2.99%, 3%, 3.01%, 3.02%, 3.03%, 3.04%, 3.05%, 3.06%, 3.07%, 3.08%, 3.09%, 3.1%, 3.11%, 3.12%, 3.13%, 3.14%, 3.15%, 3.16%, 3.17%, 3.18%, 3.19%, 3.2%, 3.21%, 3.22%, 3.23%, 3.24%, 3.25%, 3.26%, 3.27%, 3.28%, 3.29%, 3.3%, 3.31%, 3.32%, 3.33%, 3.34%, 3.35%, 3.36%, 3.37%, 3.38%, 3.39%, 3.4%, 3.41%, 3.42%, 3.43%, 3.44%, 3.45%, 3.46%, 3.47%, 3.48%, 3.49%, 3.5%, 3.51%, 3.52%, 3.53%, 3.54%, 3.55%, 3.56%, 3.57%, 3.58%, 3.59%, 3.6%, 3.61%, 3.62%, 3.63%, 3.64%, 3.65%, 3.66%, 3.67%, 3.68%, 3.69%, 3.7%, 3.71%, 3.72%, 3.73%, 3.74%, 3.75%, 3.76%, 3.77%, 3.78%, 3.79%, 3.8%, 3.81%, 3.82%, 3.83%, 3.84%, 3.85%, 3.86%, 3.87%, 3.88%, 3.89%, 3.9%, 3.91%, 3.92%, 3.93%, 3.94%, 3.95%, 3.96%, 3.97%, 3.98%, 3.99%, 4%, 4.01%, 4.02%, 4.03%, 4.04%, 4.05%, 4.06%, 4.07%, 4.08%, 4.09%, 4.1%, 4.11%, 4.12%, 4.13%, 4.14%, 4.15%, 4.16%, 4.17%, 4.18%, 4.19%, 4.2%, 4.21%, 4.22%, 4.23%, 4.24%, 4.25%, 4.26%, 4.27%, 4.28%, 4.29%, 4.3%, 4.31%, 4.32%, 4.33%, 4.34%, 4.35%, 4.36%, 4.37%, 4.38%, 4.39%, 4.4%, 4.41%, 4.42%, 4.43%, 4.44%, 4.45%, 4.46%, 4.47%, 4.48%, 4.49%, 4.5%, 4.51%, 4.52%, 4.53%, 4.54%, 4.55%, 4.56%, 4.57%, 4.58%, 4.59%, 4.6%, 4.61%, 4.62%, 4.63%, 4.64%, 4.65%, 4.66%, 4.67%, 4.68%, 4.69%, 4.7%, 4.71%, 4.72%, 4.73%, 4.74%, 4.75%, 4.76%, 4.77%, 4.78%, 4.79%, 4.8%, 4.81%, 4.82%, 4.83%, 4.84%, 4.85%, 4.86%, 4.87%, 4.88%, 4.89%, 4.9%, 4.91%, 4.92%, 4.93%, 4.94%, 4.95%, 4.96%, 4.97%, 4.98%, 4.99%, or 5% by weight of aluminum starch octenylsuccinate.

This combination of ingredients can be used in different product forms to treat various skin conditions. By way of non-limiting examples, the combination of ingredients can be formulated in an emulsion (e.g., oil in water, water in oil), a gel, a serum, a gel emulsion, a gel serum, a lotion, a mask, a scrub, a wash, a cream, or a body butter.

II. Additional UV Absorption and/or Reflective Ingredients

Contemplated herein, in some aspects, are compositions comprising one or more additional organic UV filters, UV absorption, and/or reflective ingredients (e.g., chemical and physical sunblocks) that can be used in combination with the compositions of the present disclosure including avobenzone, octocrylene, and octisalate, as well as methods for use of such compositions in protecting skin from ultraviolet radiation.

Various organic UV filters are recognized in the art for their use in protection from UV radiation and are contemplated herein. Non-limiting examples of organic UV filters which may be included in the disclosed compositions include, but are not limited to, a para-aminobenzoate derivative, a salicylate derivative, a cinnamate derivative, a benzophenone or an aminobenzophenone, an anthranillate derivative, a β,β-diphenylacrylate derivative, a benzylidenecamphor derivative, a phenylbenzimidazole derivative, a benzotriazole derivative, a triazine derivative, a bisresorcinyl triazine, an imidazoline derivative, a benzalmalonate derivative, a 4,4-diarylbutadiene derivative, a benzoxazole derivative, a merocyanine, malonitrile or a malonate diphenyl butadiene derivative, a chalcone derivative, and mixtures thereof. In some aspects, the organic UV filter is not homosalate. A sunscreen composition of the present disclosure may comprise avobenzone, octocrylene, octisalate, and 1, 2, 3, 4, 5, or more additional organic UV filters.

Non-limiting examples of chemical sunblocks that can be used include octisalate, avobenzone, octocrylene, para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate (octinoxate), isoamyl p methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidene camphor, and isopentyl 4-methoxycinnamate. Non-limiting examples of physical sunblocks include, kaolin, talc, petrolatum and metal oxides (e.g., titanium dioxide and zinc oxide).

A composition of the disclosure may include any suitable amount of one or more UV filters. In one embodiment, the composition includes about 10% to about 40%, by weight, of UV filters based on the total weight of the composition.

The one or more UV filters may include any suitable UV filter or UV filter system, including, but not limited to, solid organic lipsoluble UV filters, such as, but not limited to, butyl methoxydibenzoylmethane, and ethylhexyl trazone, liposoluble organic UV filters, such as, but not limited to, cinnamate compounds, anthranilates, salicylate compounds, dibenzoylmethane compounds, such as avobenzone, camphor compounds, 13,13-diphenylacrylate compounds, triazine compounds, benzotriazole compounds, benzalmalonate compounds (particularly those cited in U.S. Pat. No. 5,624,663), imidazoline compounds, p-aminobenzoate compounds (PABA), benzoxazole compounds (as described in patent applications EP0832642, EP1027883, EP1300137, and DE10162844), UV-filter polymers and UV-filter silicones (as described in patent application WO-93/04665), α-alkylstyrene dimers (as described in patent application DE19855649), 4,4-diarylbutadiens (as described in patent applications EP0967200, DE19746654, DE19755649, EP-A-1008586, EP1133980, and EP133981), merocyanine (as described in U.S. Pat. No. 4,195,999, WO2004/006878, WO2008/090066, WO2011113718, WO2009027258, and the documents IP COM JOURNAL No 000179675D published on Feb. 23, 2009, IP COM JOURNAL No 000182396D published on Apr. 29, 2009, IP COM JOURNAL No 000189542D published on Nov. 12, 2009, IP COM Journal No IPCOM000011179D published on Mar. 4, 2004), and their mixtures. The above documents are incorporated by reference in their entirety.

By way of non-limiting example, at least one UV filter or UV filter system may include (listed by INCI name): dibenzoylmethane compounds such as butylmethoxydibenzoylmethane (for example, as sold under the trade name PARSOL 1789® by DSM Nutritional Products, Inc.) and isopropyldibenzoylmethane; para-aminobenzoic and PABA ester compounds such as glyceryl PABA, amyldimethyl PABA, octyldimethyl PABA ethyl PABA, butyl PABA, ethyl dihydroxypropyl PABA, ethylhexyl dimethyl PABA (sold under the name ESCALOL 507@ by ISP), and glyceryl PABA; salicylic derivatives such as homosalate (sold under the commercial name EUSOLEX® HMS by Rona/EM Industries) and ethylhexyl salicylate (sold under the commercial name NEO HELIOPAN® OS by Symrise); cinnamic derivatives such as ethylhexyl methoxycinnamate (sold under the commercial name PARSOL® MCX by DSM Nutritional Products), isopropyl methoxy cinnamate, isoamyl methoxy cinnamate (sold under the commercial name NEO HELIOPAN® E 1000 by Symrise), and cinoxate, diisopropyl methylcinnamate; derivatives of β,β-diphenylacrylate such as octocrylene (sold under the commercial name UVINUL® N 539 by BASF) and etocrylene (sold under the commercial name UVINUL® N 35 by BASF); and hexyl 2-(4-diethylamino-2-hydroxybenzoyl) benzoate (sold under the commercial name UVINUL® A Plus or in the form of a mixture with octylmethoxycinnamate under the commercial name UVINUL® A+B by BASF); benzylidenecamphor derivatives such as 3-Benzylidene camphor (manufactured under the commercial name MEXORYL® SD by Chimex), 4-Methylbenzylidene camphor (sold under the commercial name EUSOLEX® 6300 by Merc), and polyacrylamidomethyl benzylidene camphor (manufactured under the commercial name MEXORYL® SW by Chimex); phenyl benzotriazole derivatives such as drometrizole trisiloxane (sold under the commercial name Silatrizole by Rhodia Chimie); triazine derivatives such as bis-ethylhexyloxyphenol methoxyphenyl triazine (sold under the commercial name TINOSORB® S by BASF), ethylhexyl triazone (sold under the commercial name UVINUL® T 150 by BASF), diethylhexyl butamido triazone (sold under the commercial name UVASORB® HEB by Sigma 3V), 2,4,6-tris(4′-amino benzalmalonate de dineopentyle)-s-triazine, 2,4,6-tris-(diisobutyle-4′-amino benzalmalonate)-s-triazine, and 2,4-bis(dineopentyle-4′-aminobenzalmalonate)-6-(4′-aminobenzoate de n-butyle)-s-triazine; triazine silicones substituted by two aminobenzoates groups such 2,4-bis-(n-butyl 4′-aminobenzalmalonate)-6-[(3-{1,3,3,3-tetramethyl-1-[(trimethyl-silyloxy]-disiloxanyl}propyl)amino]-s-triazine (and others as described in the patent EP0841341); anthranilic derivatives such as menthyl anthranilate (sold under the commercial name NEO HELIOPAN® MA by Symrise), imidazoline derivatives such as ethylhexyl dimethoxybenzylidene dioxoimidazoline propionate; benzalmalonate derivatives such as di-neopentyl 4′-methoxybenzalmalonate and polyorganosiloxane with benzalmalonate functions such as Polysilicone-15 (sold under the commercial name PARSOL® SLX by DSM Nutritional Products); derivatives of 4,4-diarylbutadiene such as 1,1-dicarboxy (2,2′-dimethyl-propyl)-4,4-diphenylbutadiene; benzoxazole derivatives such as 2,4-bis-[5-1 (dimethylpropyl)benzoxazol-2-yl-(4-phenyl)-imino]-6-(2-ethylhexyl)-imino-1,3,5-triazine (sold under the commercial name UVASORBR K2A by Sigma 3V); lipophilic merocyanine derivatives such as Octyl-5-N,N-diethylamino-2-phenysulfonyl-2,4-pentadienoate; terephthalylidene dicamphor sulfonic acid (Sold under the commercial name MEXORYL® SX by Chimex; and drometrizole trisiloxane (Sold under the commercial name MEXORYL® XL by Rhodia Chimie).

In some aspects, the compositions comprise 0.001% to 20% by weight of the one or more organic UV filters, UV absorption, and/or reflective ingredients (e.g., chemical and physical sunblocks) that can be used in combination with the compositions of the present disclosure including avobenzone, octocrylene, and octisalate. In non-limiting aspects, for example, the compositions can comprise, consist essentially of, or consist of, in their final form, for example, at least, at most, exactly, between (inclusive or exclusive), or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, or 20% by weight of the one or more organic UV filters, UV absorption, and/or reflective ingredients (e.g., chemical and physical sunblocks) that can be used in combination with the compositions of the present disclosure including avobenzone, octocrylene, and octisalate.

### III. Amounts of Ingredients

It is contemplated that the compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consist essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### IV. Vehicles

The compositions of the present invention can include or be incorporated into all types of vehicles and carriers. The vehicle or carrier can be a pharmaceutically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

### V. Structure

The compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, masks, scrubs, body butters, peels, and ointments. Variations and other structures will be apparent to the skilled artisan and are appropriate for use in the present invention.

### VI. Additional Ingredients

In addition to the combination of ingredients disclosed by the inventors, the compositions can also include additional ingredients such as cosmetic ingredients and pharmaceutical active ingredients. Non-limiting examples of these additional ingredients are described in the following subsections.

### A. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatoiretanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate), additional ingredients (iron oxides, aluminum hydroxide, and disodium stearoyl glutamate). Non-limiting examples of some of these ingredients are provided in the following subsections.

The extracts described herein can be extracts made through extraction methods known in the art and combinations thereof. Non-limiting examples of extraction methods include the use of liquid-liquid extraction, solid phase extraction, aqueous extraction, ethyl acetate, alcohol, acetone, oil, supercritical carbon dioxide, heat, pressure, pressure drop extraction, ultrasonic extraction, etc. Extracts can be a liquid, solid, dried liquid, re-suspended solid, etc.

### 1. Moisturizing Agents

Non-limiting examples of moisturizing agents that can be used with the compositions of the present disclosure include caprylyl methicone, PVP/eicosene copolymer, dimethicone, caprylyl glycol, 4-t-butylcyclohexanol, niacinamide, oligopeptide-1, Opuntia ficus-indica fruit extract, Centella asiatica meristem extract, amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

### 2. Antioxidants

Non-limiting examples of antioxidants that can be used with the compositions of the present disclosure include hydroxyacetophenone, niacinamide, oligopeptide-1, Centella asiatica meristem extract, Opuntia ficus-indica fruit extract, Alpinia galanga leaf extract, Saussurea involucrate extract acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

### 3. Structuring Agents

In other non-limiting aspects, the compositions of the present disclosure can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include ceteareth-25, disodium ethylene dicocamide, PEG-15 disulfate (CERALUTION ES) stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

### 4. Emulsifiers

In certain aspects of the present disclosure, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (see U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, cetyl PEG/PPG-10/1 dimethicone, pentaerythrityl, tetra-di-t-butyl hydroxyhydrocinnamate, polyglyceryl-4, diisostearate, polyhydroxystearate, sebacate, dimethicone/PEG-10/15 crosspolymer, dipropylene glycol, sodium citrate, tocopherol, and mixtures thereof.

### 5. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds include silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, e.g., normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, e.g., dimethicones having a viscosity of about 50 cst or less (e.g., dimethicones such as Dow Corning 200-0.5 cst Fluid). The Dow Corning Fluids are available from Dow Corning Corporation, Midland, Michigan. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Michigan.

### 6. Exfoliating Agent

Exfoliating agents include ingredients that remove dead skin cells on the skin's outer surface. These agents may act through mechanical, chemical, and/or other means. Non-limiting examples of mechanical exfoliating agents include abrasives such as pumice, silica, cloth, paper, shells, beads, solid crystals, solid polymers, etc. Non-limiting examples of chemical exfoliating agents include acids and enzyme exfoliants. Acids that can be used as exfoliating agents include, but are not limited to, glycolic acid, lactic acid, citric acid, alpha hydroxy acids, beta hydroxy acids, etc. Other exfoliating agents known to those of skill in the art are also contemplated as being useful within the context of the present invention.

### 7. Essential Oils

Essential oils include oils derived from herbs, flowers, trees, and other plants. Such oils are typically present as tiny droplets between the plant's cells and can be extracted by several method known to those of skill in the art (e.g., steam distilled, enfleurage (e.g., extraction by using fat), maceration, solvent extraction, or mechanical pressing). When these types of oils are exposed to air they tend to evaporate (e.g., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang-ylang. Other essential oils known to those of skill in the art are also contemplated as being useful within the context of the present disclosure.

### 8. Thickening Agents

Thickening agents, including thickener or gelling agents, include substances which that can increase the viscosity of a composition. Thickeners include those that can increase the viscosity of a composition without substantially modifying the efficacy of the active ingredient within the composition. Thickeners can also increase the stability of the compositions of the present invention. In certain aspects of the present invention, thickeners include xanthan gum, PVP/Eicosene copolymer, ammonium acryloyldimethyltaurate/VP copolymer (ARISTOFLEX® AVC), cetearyl alcohol, phenoxyethanol, caprylyl glycol, Ethylhexylglycerin, hexylene glycol (BOTANISTAT PF-64), Silica, hydrogenated polyisobutene, trihydroxystearin, ammonium acryloyldimethyltaurate/VP copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerythritol (e.g., CARBOPOL™ 900 series from B. F. GOODRICH).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically, these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three units.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

### 9. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, caprylyl glycol, chlorphenesin, benzyl alcohol, chlorobutanol, phenol, sorbic acid, thimerosal or combinations thereof.

### 10. Emollients

Useful emollients include the following: (a) silicone oils and modifications thereof such as linear and cyclic polydimethylsiloxanes; amino, alkyl, alkylaryl, and aryl silicone oils; (b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride; (c) waxes such as carnauba, spermaceti, beeswax, lanolin, and derivatives thereof; (d) hydrophobic plant extracts; (e) hydrocarbons such as liquid paraffins, vaseline, microcrystalline wax, ceresin, squalene, pristan and mineral oil; (f) higher fatty acids such as lauric, myristic, palmitic, stearic, behenic, oleic, linoleic, linolenic, lanolic, isostearic, arachidonic and poly unsaturated fatty acids (PUFA); (g) higher alcohols such as lauryl, cetyl, stearyl, oleyl, behenyl, cholesterol and 2-hexydecanol alcohol; (h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; (i) essential oils and extracts thereof such as mentha, jasmine, camphor, white cedar, bitter orange peel, ryu, turpentine, cinnamon, bergamot, Citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils; (j) lipids such as cholesterol, ceramides, sucrose esters and pseudo-ceramides as described in European Patent Specification No. 556,957; (k) vitamins, minerals, and skin nutrients such as vitamins A, E, and K; vitamin alkyl esters, including vitamin C alkyl esters; magnesium, calcium, and milk; (l) sunscreens such as octyl methoxyl cinnamate (Parsol MCX) and butyl methoxy benzoylmethane (Parsol 1789); (l) phospholipids; (m) polyhydric alcohols such as glycerine, propane diol and propylene glycol; and polyols such as polyethylene glycols; (n) antiaging compounds such as alpha hydroxy acids, beta hydroxy acids; and (o) mixtures of any of the foregoing components, and the like.

### 11. Tackifiers

Examples of suitable tackifiers, include, but are not limited to, aliphatic hydrocarbon resins, aromatic modified aliphatic hydrocarbon resins, hydrogenated polycyclopentadiene resins, polycyclopentadiene resins, gum rosins, gum rosin esters, wood rosins, wood rosin esters, tall oil rosins, tall oil rosin esters, polyterpenes, aromatic modified polyterpenes, terpene phenolics, aromatic modified hydrogenated polycyclopentadiene resins, hydrogenated aliphatic resin, hydrogenated aliphatic aromatic resins, hydrogenated terpenes and modified terpenes, hydrogenated rosin acids, hydrogenated rosin esters, polyisoprene, partially or fully hydrogenated polyisoprene, polybutenediene, partially or fully hydrogenated polybutenediene, and the like. As is evidenced by some of the cited examples, the tackifier may be fully or partially hydrogenated. The tackifier may also be non-polar. (Non-polar meaning that the tackifier is substantially free of monomers having polar groups. Preferably, the polar groups are not present, however, if they are present, they are preferably present in an amount of up to about 5% by weight, preferably up to about 2% by weight, and more preferably up to about 0.5% by weight).

### 12. Colorants

The compositions of the present invention also contain at least one cosmetically acceptable colorant such as a pigment or dyestuff. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface-treated within the scope of the present invention but are not limited to treatments such as silicones, perfluorinated compounds, lecithin, and amino acids.

### 13. Surfactants

Surfactants useful as the surfactant components in the compositions of the present invention include PEG-15 disulfate, glyceryl stearate, nonionic, anionic, cationic, and amphoteric (zwitterionic) surfactants and may be used in combination with each other.

### 14. pH Adjustors

The pH adjustors, include inorganic and organic acids and bases and in particular aqueous ammonia, citric acid, phosphoric acid, acetic acid, sodium hydroxide, lactic acid, levulinic acid, glycolic acid, tartaric acid, malic acid, pyrrolidonecarboxylic acid (PCA), succinic acid, citric acid, glutamic acid, 2-amino-2-methyl-1-propanol (AMP), and triethanolamine (TEA).

15. Reducing agents

Suitable reducing agents include, but are not limited to, thiourea, salts (such as sodium salts) of thiosulfate, sulfite, bisulfite, metabisulfite, borohydride, and hypophosphite, ascorbic acid and salts, esters, and derivatives thereof (e.g., ascorbyl palmitate and ascorbyl polypeptide), and tocopherols and salts, esters, and derivatives thereof (e.g., tocopherol acetate). Other reducing agents are listed on pages 1655-56 of the INCI Handbook.

### 16. Fragrances

The compositions disclosed herein may optionally include a fragrance. Examples of possible fragrances include natural oils or naturally derived materials, and synthetic fragrances such as hydrocarbons, alcohols, aldehydes, ketones, esters, lactones, ethers, nitriles, and polyfunctionals.

Non-limiting examples of natural oils include the following: basil (Ocimum basilicum) oil, bay (Pimento acris) oil, bee balm (Monarda didyma) oil, bergamot (Citrus aurantium bergamia) oil, cardamom (Elettaria cardamomum) oil, cedarwood (Cedrus atlantica) oil, chamomile (Anthemis nobilis) oil, cinnamon (Cinnamomum cassia) oil, citronella (Cymbopogon nardus) oil, clary (Salvia sclarea) oil, clove (Eugenia caryophyllus) oil, cloveleaf (Eufenia caryophyllus) oil, Cyperus esculentus oil, cypress (Cupressus sempervirens) oil, Eucalyptus citriodora oil, Geranium maculatum oil, ginger (Zingiber officinale) oil, grapefruit (Citrus grandis) oil, hazel (Corylus avellana) nut oil, jasmine (Jasminum officinale) oil, Juniperus communis oil, Juniperus oxycedrus tar, Juniperus virginiana oil, kiwi (Actinidia chinensis) water, lavandin (Lavandula hybrida) oil, lavender (Lavandula angustifolia) oil, lavender (Lavandula angustifolia) water, lemon (Citrus medica limonum) oil, lemongrass (Cymbopogon schoenanthus) oil, lime (Citrus aurantifolia) oil, linden (Tilia cordata) oil, linden (Tilia cordata) water, mandarin orange (Citrus nobilis) oil, nutmeg (Myristica fragrans) oil, orange (Citrus aurantium dulcis) flower oil, orange (Citrus aurantium dulcis) oil, orange (Citrus aurantium dulcis) water, patchouli (Pogostemon cablin) oil, peppermint (Menthe piperita) oil, peppermint (Menthe peperita) water, rosemary (Rosmarinus officinalis) oil, rose oil, rose (Rosa damascena) extract, rose (Rosa multiflora) extract, rosewood (Aniba rosaeodora) extract, sage (Salvia officinalis) oil, sandalwood (Santalum album) oil, spearmint (Menthe viridis) oil, tea tree (Melaleuca alternifolia) oil, and ylang (Cananga odorata) oil.

Some non-limiting examples of synthetic hydrocarbon fragrances include caryophyllene, β-farnesene, limonene, α-pinene, and, β-pinene. Some non-limiting examples of synthetic alcohol fragrances include bacdanol, citronellol, linalool, phenethyl alcohol, and α-terpineol (R=H). Some non-limiting examples of synthetic aldehyde fragrances include 2-methyl undecanal, citral, hexyl cinnamic aldehyde, isocycolcitral, lilial, and 10-undecenal. Some non-limiting examples of synthetic ketone fragrances include cashmeran, α-ionone, isocyclemone E, koavone, muscone, and tonalide. Some non-limiting examples of synethetic ester fragrances include benzyl acetate, 4-t-butylcyclohexyl acetate (cis and trans), cedryl acetate, cyclacet, isobornyl acetate, and α-terpinyl acetate (R=acetyl). Some non-limiting examples of synthetic lactone fragrances include coumarin, jasmine lactone, muskalactone, and peach aldehyde. Some non-limiting examples of synthetic ether fragrances include ambroxan, anther, and galaxolide. Some non-limiting examples of synthetic nitrile fragrances include cinnamonitrile and gernonitrile. Finally, some non-limiting examples of synthetic polyfunctional fragrances include amyl salicylate, isoeugenol, hedione, heliotropine, lyral, and vanillin.

### 17. Foaming Agents

The foaming agents include, for example, sodium lauryl sulfate, sodium lauroyl sarcosine, sodium alkyl sulfosuccinates, sodium coconut oil fatty acid monoglycerol sulfonates, sodium α-olefin sulfonates, N-acylamino acid salts such as N-acyl glutamate, 2-alkyl-N-carboxymethyl-N-hydroxyethylimidazolinium betaine, maltitol fatty acid esters, sucrose fatty acid esters, polyglycerol fatty acid esters, fatty acid diethanolamides, polyoxyethylene sorbitan monostearate, polyoxyethylene hydrogenated castor oil and polyoxyethylene fatty acid esters. These foaming agents are usable either alone or in combination of two or more of them.

### 18. Tanning Agents

Suitable tanning agents include, without limitation, alpha-hydroxy aldehydes and ketones, glyceraldehyde and related alcohol aldehydes, various indoles, imidazoles and derivatives thereof, and various approved pigmentation agents. Other suitable tanning agents include, without limitation, methyl glyoxal, glycerol aldehyde, erythrulose, alloxan, 2,3-dihydroxysuccindialdehyde, 2,3-dimethoxysuccindialdehyde, 2-amino-3-hydroxysuccindialdehyde and 2-benzylamino-3-hydroxysuccindialdehyde.

### 19. Astringents

Suitable astringents include, without limitation, aluminum citrate, aluminum lactate, extracts of birch, extracts of coffee, extracts of evening primrose, extracts of grape, extracts of henna, extracts of ivy, extracts of lemon, extracts of witch hazel, Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof. What is meant by an extract is either the whole fruit, bean, and/or plant or select constituents of such fruit, bean, and/or plant.

### 20. Antiseptics

Suitable antiseptics include, without limitation, methyl, ethyl, propyl, or butyl ester of p-oxybenzoic acid, phenoxyethanol, o-phenylphenol, dehydroacetic acid, or salts thereof, p-cresol, m-cresol, o-chlor-m-xylenol, peppermint oil, Echinacea, bloodroot, cayenne, tea tree oil, wild bergamont, chaparral, stinging metal, bay, myrrh, rhatany bark, toothache tree, calendula, chamomile, mupirocin, neomycin sulfate, bacitracin, polymyxin B, I-ofloxacin, tetracyclines (chlortetracycline hydrochloride, oxytetracycline hydrochloride and tetracycline hydrochloride), clindamycin phosphate, gentamicin sulfate, benzalkonium chloride, benzethonium chloride, hexylresorcinol, methylbenzethonium chloride, phenol, quaternary ammonium compounds, triclocarbon, triclosan, and tea tree oil.

### 21. Deodorants and Antiperspirants

Suitable antiperspirants and deodorants include, without limitation, zinc salts such as zinc sulfate and zinc chloride, glycinates such as aluminum zirconium glycinate, aluminum chlorohydrate, aluminum zirconium tetrachlorohydrex, zinc carbonate, orthophenylphenol, and quaternary ammonium compounds such as dimethyl benzyl ammonium chloride and hexamethonium chloride.

### 22. Lighteners

Examples of skin lighteners include, without limitation, hydroquinone, kojic acid, licorice and/or its derivatives, ascorbic acid and/or its derivatives, arbutin, bearberry extract, Glycyrrhiza glabra and its derivatives, Chlorella vulgaris extract, perilla extract, coconut fruit extract, and/or other depigmenting agents.

### 23. Biocides

Examples of biocides include, without limitation, triclosan, 3,4,4′-trichlorocarbanilide (triclocarban); 3,4,4′-trifluoromethyl-4,4′-dichlorocarbanilide (cloflucarban); 5-chloro-2-methyl-4-isothiazolin-3-one; iodopropynlbutylcarbamate; 8-hydroxyquinoline; 8-hydroxyquinoline citrate; 8-hydroxyquinoline sulfate; 4-chloro-3,5-xylenol (chloroxylenol); 2-bromo-2-nitropropane-1,3-diol; diazolidinyl urea; butoconazole; nystatin; terconazole; nitrofurantoin; phenazopyridine; acyclovir; clortrimazole; chloroxylenol; chlorhexidine; miconazole; terconazole; butylparaben; ethylparaben; methylparaben; methylchloroisothiazoline; methylisothiazoline; a mixture of 1,3-bis(hydroxymethyl)-5,5-dimethylhydantoin and 3-iodo-2-propynyl butyl carbamate; oxyquinoline; EDTA; tetrasodium EDTA; p-hydroxyl benzoic acid ester; alkyl pyridinum compounds; coco phosphatidyl PG-dimonium chloride; chlorhexidine gluconate; chlorhexidine digluconate; chlorhexidine acetate; chlorhexidine isethionate; chlorhexidine hydrochloride; benzalkonium chloride; benzethonium chloride; polyhexamethylene biguanide; and mixtures thereof.

### B. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### VII. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1

### (Testing Vehicles)

Tables 1 and 2 describe generic skin testing formulations in which the sunscreen agents and aluminum starch octenylsuccinate can be incorporated into to determine the efficacy and stability of the composition to protect skin from ultraviolet radiation, reduce or prevent sunburn, and/or reduce or prevent melanin overproduction due to sun exposure and/or UV radiation exposure.

TABLE 1*

| | % Concentration |
|---|---|
| Ingredient | (by weight) |
| Phase A | |
| Water | 84.80 |
| Xanthan gum | 0.1 |
| M-paraben | 0.15 |
| P-paraben | 0.1 |
| Citric acid | 0.1 |
| Phase B | |
| Cetyl alcohol | 4.0 |
| Glyceryl stearate + PEG 100 | 4.0 |
| Octyl palmitate | 4.0 |
| Dimethicone | 1.0 |
| Tocopheryl acetate | 0.2 |
| Phase C | |
| Active Ingredient(s)** | 2.0 |
| TOTAL | 100 |

*Procedure for making composition: Sprinkle Xanthan gum in water and mix for 10 min. Subsequently, add all ingredients in phase A and heat to 70-75° C. Add all items in phase B to separate beaker and heat to 70-75° C. Mix phases A and B at 70-75° C. Continue mixing and allow composition to cool to 30° C. Subsequently, add phase C ingredient while mixing.
**The active ingredients identified throughout this specification can be incorporated into composition as the active ingredient. The active ingredients can be individually used or combined in this composition. The concentration ranges of the active ingredients (or combination of active ingredients) can be modified as desired or needed by increasing or decreasing the amount of water. Example active ingredients which may be included include avobenzone, octocrylene, octisalate, and aluminum starch octenylsuccinate.

TABLE 2*

| | % Concentration |
|---|---|
| Ingredient | (by weight) |
| Phase A | |
| Water | 78.6 |
| M-paraben | 0.2 |
| P-paraben | 0.1 |
| Na₂ EDTA | 0.1 |
| Shea butter | 4.5 |
| Petrolatum | 4.5 |
| Glycerin | 4.0 |
| Propylene Glycol | 2.0 |
| Finsolve TN | 2.0 |
| Phase B | |
| Sepigel 305 | 2.0 |
| Phase C | |
| Active Ingredient(s)** | 2.0 |
| TOTAL | 100 |

*Add ingredients in phase A to beaker and heat to 70-75° C. while mixing. Subsequently, add the phase B ingredient with phase A and cool to 30° C. with mixing. Subsequently, add phase C ingredient while mixing.
**The active ingredients identified throughout this specification can be incorporated into composition as the active ingredient. The active ingredients can be individually used or combined in this composition. The concentration ranges of the active ingredients (or combination of active ingredients) can be modified as desired or needed by increasing or decreasing the amount of water. Example active ingredients which may be included include avobenzone, octocrylene, octisalate, and aluminum starch octenylsuccinate.

### Example 2

### (Exemplary Formulations)

Formulations 1-3 having the ingredients disclosed herein were prepared as topical skin compositions. In some instances, the topical skin compositions can be prepared as a liquid, serum, cream, gel, cream gel, ointment, spray, emulsion, or gel emulsion. Formulations 1-3 in Tables 3-5 are examples of sunscreen compositions of the present disclosure prepared as lotions.

TABLE 3*
Formulation 1

| Ingredients | % (w/w) |
|---|---|
| Octisalate | 4.50 |
| Capryl methicone | 1.50 |
| Avobenzone | 3.00 |
| Octocrylene | 9.00 |
| Ceralution ES | 2.00 |
| PVP-eicosene copolymer | 0.50 |
| Glyceryl stearate | 1.00 |
| Beeswax | 1.00 |
| Water | 66.55 |
| Glycerin | 2.00 |
| Disodium EDTA | 0.10 |
| Hydroxyacetophenone | 0.25 |
| Propanediol | 3.00 |
| Xanthan gum | 0.10 |
| Aristoflex AVC | 1.00 |
| Euxyl PE 9010 | 0.70 |
| Dimethicone silicone HL 88 | 0.80 |
| Silica MSS-500 | 1.00 |
| Silica | 1.00 |
| Aluminium starch octenyl succinate | 1.00 |
| TOTAL | 100 |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.

TABLE 4*
Formulation 2

| Ingredients | % (w/w) |
|---|---|
| Octisalate | 4.50 |
| Capryl methicone | 1.50 |
| Avobenzone | 3.00 |
| Octocrylene | 9.00 |
| Ceralution ES | 2.00 |
| PVP-eicosene copolymer | 0.50 |
| Glyceryl stearate | 1.00 |
| Beeswax | 1.00 |
| Water | 61.10 |
| Glycerin | 2.00 |
| Disodium EDTA | 0.10 |
| Hydroxyacetophenone | 0.25 |
| Propanediol | 3.00 |
| Xanthan gum | 0.10 |
| Aristoflex AVC | 1.00 |
| Water | 3.00 |
| Niacinamide | 1.00 |
| Euxyl PE 9010 | 0.70 |
| SPI EVO-LOCK RES (encapsulated resveratrol) | 0.15 |
| Dimethicone silicone HL 88 | 0.80 |
| Oligopeptide-1 | 0.30 |
| Opuntia ficus-indicia fruit extract | 1.00 |
| Silica MSS-500 | 1.00 |
| Silica | 1.00 |
| Aluminum starch octenylsuccinate | 1.00 |
| TOTAL | 100 |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.

TABLE 5*
Formulation 3

| Ingredients | % (w/w) |
|---|---|
| Octisalate | 4.50 |
| Capryl methicone | 1.50 |
| Avobenzone | 3.00 |
| Octocrylene | 9.00 |
| Ceralution ES | 2.00 |
| PVP-eicosene copolymer | 0.50 |
| Cetearyl alcohol | 1.20 |
| Glyceryl stearate | 1.50 |
| Beeswax | 1.00 |
| Water | 54.00 |
| Glycerin | 2.00 |
| Disodium EDTA | 0.10 |
| Hydroxyacetophenone | 0.25 |
| Propanediol | 3.00 |
| Xanthan gum | 0.10 |
| Aristoflex AVC | 1.30 |
| Water | 0.50 |
| Potassium hydroxide 45% | 0.10 |
| Botanistat PF064 | 0.40 |
| Symocide PS | 0.40 |
| Symbiocel BC 10015 | 1.00 |
| Aluminum starch octenylsuccinate | 1.00 |
| Silica MSS-500 | 1.00 |
| Silica | 1.00 |
| Water | 3.50 |
| Calcium ketogluconate | 0.50 |
| Dimethicone silicone HL 88 | 0.80 |
| Kollaren | 0.50 |
| Gentelia asiatica meristem | 0.25 |
| Pronalen Silymarin | 1.00 |
| 4-T-Butylcyclohexanol | 1.00 |
| Sodium PCA | 0.10 |
| Alpinia galanga leaf extract | 1.00 |
| Saussurea involucrata Ex. & glycerin & water | 1.00 |
| TOTAL | 100 |

### Example 3

### (Sun Protection Factor Determination)

The sun protection factor (SPF) was determined for Formulations 1-3 provided in Tables 3-5, which represent sunscreen compositions of the present disclosure prepared as lotions. SPF is a measure of how much UV radiation (e.g., solar energy) is required to produce sunburn on protected skin (e.g., in the presence of sunscreen compositions) relative to the amount of UV radiation (e.g., solar energy) required to produce sunburn on unprotected skin (e.g., in the absence of sunscreen compositions). In general, as SPF value increases, sunburn protection increases. The SPF values for Formulations 1-3 from Tables 3-5 were as follows: Formulation 1 (Table 3), SPF 43; Formulation 2, SPF 40 (Table 4); Formulation 3, unknown (Table 5).

### Example 4

### (Additional Assays)

Assays that can be used to determine the efficacy of any one of the ingredients or any combination of ingredients or compositions having said combination of ingredients disclosed throughout the specification and claims can be determined by methods known to those of ordinary skill in the art. The following are non-limiting assays that can be used in the context of the present disclosure. It should be recognized that other testing procedures can be used, including, for example, objective and subjective procedures.

Collagen Stimulation Assay: A collagen stimulation assay can be used to determine the ability of any one of the active ingredients, combination of ingredients, or compositions having said combinations disclosed in the specification to increase expression of procollagen-1, a precursor to collagen. Collagens (types I, II, III, IV and V) can be synthesized as precursor molecules called procollagens. These precursor molecules can contain additional peptide sequences, usually called “propeptides”, at both the amino-terminal and the carboxy-terminal ends. During cellular expression and secretion, procollagens can be assembled in the trimeric form and then cleaved at specific N- and C-terminal sites by specific endopeptidases, generating three fragments: procollagen-1 N-terminal propeptide (PINP), Type I collagen, and procollagen-1 carboxy-terminal propeptide (PICP).

The function of the propeptides is to facilitate the winding of procollagen molecules into a triple-helical conformation within the endoplasmic reticulum. The propeptides can be cleaved off from the collagen triple helix molecule during its secretion, after which the triple helix collagens polymerize into extracellular collagen fibrils. Thus, the amount of the free propeptides reflects stoichiometrically the amount of collagen molecules synthesized (a relationship analogous to that between the carboxy-terminal peptide of proinsulin and the endogenously produced insulin). Collagen is an extracellular matrix protein critical for skin structure. Increased synthesis of collagen helps improve skin firmness and elasticity.

Quantitative detection of PICP in fibroblast cell extracts and culture supernatants can be performed with an enzyme immunoassay kit (e.g., Takara #MK101) to assess the effects of the ingredients on the synthesis of PICP in skin. This bioassay can be used to examine effects on the production of procollagen peptide (a precursor to collagen) by human epidermal fibroblasts. The endpoint of this assay can be a spectrophotometric measurement that reflects the presence of procollagen peptide and cellular viability. The assay employs the quantitative sandwich enzyme immunoassay technique whereby a monoclonal antibody specific for procollagen peptide was pre-coated onto a microplate. Standards and samples can be pipetted into the wells and any procollagen peptide present was bound by the immobilized antibody. After washing away any unbound substances, an enzyme-linked polyclonal antibody specific for procollagen peptide can be added to the wells. Following a wash to remove any unbound antibody-enzyme reagent, a substrate solution can be added to the wells and color was developed in proportion to the amount of procollagen peptide bound in the initial step. Color development was stopped and the intensity of the color at 450 nm was measured using a microplate reader.

For generation of samples and controls, subconfluent normal human adult epidermal fibroblasts (Cascade Biologics) can be cultivated in standard DMEM growth medium with 10% fetal bovine serum (Mediatech) at 37° C. in 10% CO₂. The cells can be treated with each of the tested ingredients and controls for 3 days. Following incubation, cell culture medium can be collected and the amount of Type I procollagen peptide secretion was quantified using the sandwich enzyme linked immuno-sorbant assay (ELISA) from Takara (#MK101) as explained above.

Elastin Stimulation Assay: Elastin is a connective tissue protein that helps skin resume shape after stretching or contracting. Elastin is also an important load-bearing protein used in places where mechanical energy is required to be stored. Elastin is made by linking many soluble tropoelastin protein molecules, in a reaction catalyzed by lysyl oxidase. Elastin secretion and elastin fibers can be monitored in cultured human fibroblasts by staining of cultured human fibroblasts using immunofluorescent antibodies directed against elastin by a direct ELISA sandwich method. A Meso Scale Discovery system SECTOR 2400 Imaging system can be used to analyze the results. Changes in elastin secretion and elastin fibers caused by one or more ingredients in the composition can be determined by incubating cultured human fibroblasts with the active ingredient for a period of time before probing the cells or a lysate thereof with antibodies directed against elastin.

Laminin Stimulation Assay: Laminin is a major protein in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin is a structural glycoprotein located in the DEJ. Together with fibronectin, laminin is considered the glue that holds the cells together, and both are secreted by dermal fibroblasts to help facilitate intra- and inter-cellular adhesion of the epidermal calls to the DEJ.

Laminin secretion can be monitored by quantifying laminin in cell supernatants of cultured human fibroblasts treated for 3 days with culture medium with or without 1.0% final concentration of the test ingredient(s). Following incubation, laminin content can be measured using immunofluorescent antibodies directed against each protein in an enzyme linked immuno-sorbant assay (ELISA).

Fibronectin Stimulation Assay: Fibronectin is a major protein in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Fibronectin is a structural glycoprotein located in the DEJ. Together with laminin, fibronectin is considered the glue that holds the cells together, and both are secreted by dermal fibroblasts to help facilitate intra- and inter-cellular adhesion of the epidermal calls to the DEJ.

Fibronectin secretion can be monitored by quantifying fibronectin in cell supernatants of cultured human fibroblasts treated for 3 days with culture medium with or without 1.0% final concentration of the test ingredient(s). Following incubation, fibronectin content can be measured using immunofluorescent antibodies directed against each protein in an enzyme linked immuno-sorbant assay (ELISA).

Tumor Necrosis Factor Alpha (TNF-α) Assay: The prototype ligand of the TNF superfamily, TNF-α, is a pleiotropic cytokine that plays a central role in inflammation. Increase in its expression is associated with an up regulation in pro-inflammatory activity. This bioassay can be used to analyze the effect of any one of the active ingredients, combination of ingredients, or compositions having said combinations disclosed in the specification on the production of TNF-α by human epidermal keratinocytes. The endpoint of this assay can be a spectrophotometric measurement that reflects the presence of TNF-α and cellular viability. The assay employs the quantitative sandwich enzyme immunoassay technique whereby a monoclonal antibody specific for TNF-α has been pre-coated onto a microplate. Standards and samples can be pipetted into the wells and any TNF-α present is bound by the immobilized antibody. After washing away any unbound substances, an enzyme-linked polyclonal antibody specific for TNF-α can be added to the wells. Following a wash to remove any unbound antibody-enzyme reagent, a substrate solution can be added to the wells and color develops in proportion to the amount of TNF-α bound in the initial step using a microplate reader for detection at 450 nm. The color development can be stopped and the intensity of the color can be measured. Subconfluent normal human adult keratinocytes (Cascade Biologics) cultivated in EPILIFE™ standard growth medium (Cascade Biologics) at 37° C. in 5% CO₂, can be treated with phorbol 12-myristate 13-acetate (PMA, 10 ng/ml, SIGMA CHEMICAL, #P1585-1 MG) and any one of the active ingredients, combination of ingredients, or compositions having said combinations disclosed in the specification for 6 hours. PMA has been shown to cause a dramatic increase in TNF-α secretion which peaks at 6 hours after treatment. Following incubation, cell culture medium can be collected and the amount of TNF-α secretion quantified using a sandwich enzyme linked immuno-sorbant assay (ELISA) from R&D Systems (#DTA00C).

Antioxidant (AO) Assay: An in vitro bioassay that measures the total anti-oxidant capacity of any one of the ingredients, combination of ingredients, or compositions having said combinations disclosed in the specification. The assay relies on the ability of antioxidants in the sample to inhibit the oxidation of ABTS® (2,2′-azino-di-[3-ethylbenzthiazoline sulphonate]) to ABTS® by metmyoglobin. The antioxidant system of living organisms includes enzymes such as superoxide dismutase, catalase, and glutathione peroxidase; macromolecules such as albumin, ceruloplasmin, and ferritin; and an array of small molecules, including ascorbic acid, α-tocopherol, β-carotene, reduced glutathione, uric acid, and bilirubin. The sum of endogenous and food-derived antioxidants represents the total antioxidant activity of the extracellular fluid. Cooperation of all the different antioxidants provides greater protection against attack by reactive oxygen or nitrogen radicals, than any single compound alone. Thus, the overall antioxidant capacity may give more relevant biological information compared to that obtained by the measurement of individual components, as it considers the cumulative effect of all antioxidants present in plasma and body fluids. The capacity of the antioxidants in the sample to prevent ABTS® oxidation is compared with that of Trolox, a water-soluble tocopherol analogue, and is quantified as molar Trolox equivalents. Anti-Oxidant capacity kit #709001 from CAYMAN CHEMICAL (Ann Arbor, Michigan USA) can be used as an in vitro bioassay to measure the total anti-oxidant capacity of each of any one of the active ingredients, combination of ingredients, or compositions having said combinations disclosed in the specification. The protocol can be followed according to manufacturer recommendations.

ORAC Assay: Oxygen Radical Absorption (or Absorbance) Capacity (ORAC) of any one of the active ingredients, combination of ingredients, or compositions having said combinations disclosed in the specification can also be assayed by measuring the antioxidant activity of such ingredients or compositions. Antioxidant activity indicates a capability to reduce oxidizing agents (oxidants). This assay quantifies the degree and length of time it takes to inhibit the action of an oxidizing agent, such as oxygen radicals, that are known to cause damage to cells (e.g., skin cells). The ORAC value of any one of the active ingredients, combination of ingredients, or compositions having said combinations disclosed in the specification can be determined by methods known to those of ordinary skill in the art (see U.S. Publication Nos. 2004/0109905 and 2005/0163880; and commercially available kits such as Zen-Bio ORAC Anti-oxidant Assay kit (#AOX-2)). The Zen-Bio ORAC Anti-oxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

Matrix Metalloproteinase 1 Enzyme Activity (MMP-1) Assay: MMPs are extracellular proteases that play a role in many normal and disease states by virtue of their broad substrate specificity. MMP-1 substrates include collagen IV. The Molecular Probes Enz/Chek Gelatinase/Collagenase Assay kit (#E12055), can be used to detect MMP-1 protease activity, and utilizes a fluorogenic gelatin substrate and tests proteolytic cleavage of the substrate by purified MMP-1 enzyme. Upon proteolytic cleavage of the substrate, bright green fluorescence is revealed and can be monitored using a fluorescent microplate reader to measure enzymatic activity. Test materials can be incubated in the presence or absence of the purified enzyme and substrate to determine their protease inhibitor capacity.

Matrix Metalloproteinase 3 and 9 Enzyme Activity (MMP3; MMP9) Assay: An in vitro matrix metalloprotease (MMP) inhibition assay. MMPs are extracellular proteases that play a role in many normal and disease states by virtue of their broad substrate specificity. MMP3 substrates include collagens, fibronectins, and laminin; while MMP9 substrates include collagen VII, fibronectins and laminin. Using Colorimetric Drug Discovery kits from BioMol International for MMP3 (AK-400) and MMP-9 (AK-410), this assay is designed to measure protease activity of MMPs using a thiopeptide as a chromogenic substrate (Ac-PLG-[2-mercapto-4-methyl-pentanoyl]-LG-OC2H5)5,6. The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, which can be detected by its absorbance at 412 nm (E=13,600 M-1 cm-1 at pH 6.0 and above 7). The active ingredients, any one of the combination of ingredients, or compositions having said combinations disclosed in the specification can be assayed.

Cyclooxygenase (COX) Assay: An in vitro cyclooxygenase-1 and -2 (COX-1, -2) inhibition assay. COX is a bifunctional enzyme exhibiting both cyclooxygenase and peroxidase activities. The cyclooxygenase activity converts arachidonic acid to a hydroperoxy endoperoxide (Prostaglandin G2; PGG2) and the peroxidase component reduces the endoperoxide (Prostaglandin H2; PGH2) to the corresponding alcohol, the precursor of prostaglandins, thromboxanes, and prostacyclins. This COX Inhibitor screening assay measures the peroxidase component of cyclooxygenases. The peroxidase activity is assayed colorimetrically by monitoring the appearance of oxidized N,N,N′,N′-tetramethyl-p-phenylenediamine (TMPD). This inhibitor screening assay includes both COX-1 and COX-2 enzymes in order to screen isozyme-specific inhibitors. The Colormetric COX (ovine) Inhibitor screening assay (#760111, CAYMAN CHEMICAL) can be used to analyze the effects of each of the active ingredients, any one of the combination of ingredients, or compositions having said combinations disclosed in the specification on the activity of purified cyclooxygnase enzyme (COX-1 or COX-2). According to manufacturer instructions, purified enzyme, heme and test extracts can be mixed in assay buffer and incubated with shaking for 15 min at room temperature. Following incubation, arachidonic acid and colorimetric substrate can be added to initiate the reaction. Color progression can be evaluated by colorimetric plate reading at 590 nm. The percent inhibition of COX-1 or COX-2 activity can be calculated compared to non-treated controls to determine the ability of test extracts to inhibit the activity of purified enzyme.

Lipoxygenase (LO) Assay: An in vitro lipoxygenase (LO) inhibition assay. LOs are non-heme iron-containing dioxygenases that catalyze the addition of molecular oxygen to fatty acids. Linoleate and arachidonate are the main substrates for LOs in plants and animals. Arachadonic acid may then be converted to hydroxyeicosotrienenoic (HETE) acid derivatives, that are subsequently converted to leukotrienes, potent inflammatory mediators. This assay provides an accurate and convenient method for screening lipoxygenase inhibitors by measuring the hydroperoxides generated from the incubation of a lipoxygenase (5-, 12-, or 15-LO) with arachidonic acid. The Colorimetric LO Inhibitor screening kit (#760700, CAYMAN CHEMICAL) can be used to determine the ability of each of the active ingredients, any one of the combination of ingredients, or compositions having said combinations disclosed in the specification to inhibit enzyme activity. Purified 15-lipoxygenase and test ingredients can be mixed in assay buffer and incubated with shaking for 10 min at room temperature. Following incubation, arachidonic acid can be added to initiate the reaction and the mixtures can be incubated for an additional 10 min at room temperature. Colorimetric substrate can be added to terminate catalysis and color progression can be evaluated by fluorescence plate reading at 490 nm. The percent inhibition of lipoxyganse activity can be calculated compared to non-treated controls to determine the ability of each of the active ingredients, any one of the combination of ingredients, or compositions having said combinations disclosed in the specification to inhibit the activity of purified enzyme.

Lysyl Oxidase Assay: A lysyl oxidase assay can be performed on skin cells (e.g., epidermal keratinocytes, fibroblasts, and/or dermal endothelial cells) to determine the ability of any one of the active ingredients, combination of ingredients, or compositions having said combinations disclosed in the specification to stimulate expression of lysyl oxidase in skin. Lysyl oxidase can catalyze crosslinking of elastin and collagens, thereby providing for a more structurally rigid matrix for skin. By increasing expression of lysyl oxidase, increased cross-linking of elastin and collagens can occur, which can be beneficial in reducing the appearance of fine lines, wrinkles, sagging skin, and/or non-elastic skin.

Elastase Assay: ENZCHEK® Elastase Assay (Kit #E-12056) from MOLECULAR PROBES (Eugene, Oregon USA) can be used as an in vitro enzyme inhibition assay for measuring inhibition of elastase activity for each of the active ingredients, any one of the combination of ingredients, or compositions having said combinations disclosed in the specification. The ENZCHEK kit contains soluble bovine neck ligament elastin that can be labeled with dye such that the conjugate's fluorescence can be quenched. The non-fluorescent substrate can be digested by elastase or other proteases to yield highly fluorescent fragments. The resulting increase in fluorescence can be monitored with a fluorescence microplate reader. Digestion products from the elastin substrate have absorption maxima at ˜505 nm and fluorescence emission maxima at ˜515 nm. The peptide, N-methoxysuccinyl-Ala-Ala-Pro-Val-chloromethyl ketone, can be used as a selective, collective inhibitor of elastase when utilizing the ENZCHEK ELASTASE ASSAY KIT for screening for elastase inhibitors.

Mushroom tyrosinase activity assay: In mammalian cells, tyrosinase catalyzes two steps in the multi-step biosynthesis of melanin pigments from tyrosine (and from the polymerization of dopachrome). Tyrosinase is localized in melanocytes and produces melanin (aromatic quinone compounds) that imparts color to skin, hair, and eyes. Purified mushroom tyrosinase (from SIGMA) can be incubated with its substrate L-Dopa (from FISHER) in the presence or absence of each of the active ingredients, any one of the combination of ingredients, or compositions having said combinations disclosed in the specification. Pigment formation can be evaluated by colorimetric plate reading at 490 nm. The percent inhibition of mushroom tyrosinase activity can be calculated compared to non-treated controls to determine the ability of test ingredients or combinations thereof to inhibit the activity of purified enzyme. Test extract inhibition was compared with that of kojic acid (SIGMA).

B16 Pigmentation Assay: Melanogenesis is the process by which melanocytes produce melanin, a naturally produced pigment that imparts color to skin, hair, and eyes. Inhibiting melanogenesis is beneficial to prevent skin darkening and lighten dark spots associated with aging. This bioassay utilizes B16-F1 melanocytes (ATCC), an immortalized mouse melanoma cell line, to analyze the effect of compounds on melanogenesis. The endpoint of this assay is a spectrophotometric measurement of melanin production and cellular viability. B16-F1 melanocytes, can be cultivated in standard DMEM growth medium with 10% fetal bovine serum (MEDIATECH) at 37° C. in 10% CO₂ and then treated with any one of the active ingredients, combination of ingredients, or compositions having said combinations disclosed in the specification for 6 days. Following incubation, melanin secretion is measured by absorbance at 405 nm and cellular viability is quantified

MELANODERM™ Assay: In other non-limiting aspects, the efficacy of each of the active ingredients, any one of the combination of ingredients, or compositions having said combinations disclosed in the specification can be evaluated by using a skin analog, such as, for example, MELANODERM™. Melanocytes, one of the cells in the skin analog, stain positively when exposed to L-dihydroxyphenyl alanine (L-DOPA), a precursor of melanin. The skin analog, MELANODERM™, can be treated with a variety of bases containing each of the active ingredients, any one of the combination of ingredients, or compositions having said combinations disclosed in the specification or with the base alone as a control. Alternatively, an untreated sample of the skin analog can be used as a control.

Production of Ceramides: Ceramides in cell or tissue samples can be labeled with a mouse monoclonal antibody anti-ceramide (ENZO LIFE SCIENCE, ref ALX-804-196 clone MID15B4) diluted to 1/50 for 2 hours at room temperature with an amplifier system biotin/streptavidin. Video microscope observation can be performed to view ceramides (pink stain).

Production of Filaggrin: Changes in the production of filaggrin in keratinocytes due to each of the active ingredients, any one of the combination of ingredients, or compositions having said combinations disclosed in the specification can be measured. Filaggrin is the precursor to Natural Moisturizing Factor (NMF) in the skin. Increased NMF increases the moisture content of the skin. Filaggrin production in treated and non-treated keratinocytes can be determined using a bioassay that analyzes filaggrin concentration in keratinocyte cell lysates. A non-limiting example of a bioassay that can be used to quantify filaggrin production is the PROTEINSIMPLE® SIMON™ western blotting protocol. For each sample, normal human epidermal keratinocytes (NHEK) are grown in EPI-200-MATTEK EPILIFE® growth media with calcium from Life Technologies (M-EP-500-CA). NHEK are incubated in growth medium overnight at 37° C. in 5% CO₂ prior to treatment. NHEK are then incubated in growth medium with 1% test compound/extract or no compound/extract (negative control) for 24 to 36 hours. The NHEK can then be washed, collected, and stored on ice or colder until lysed on ice using a lysis buffer and sonication. The protein concentrations of the samples can be determined and used to normalize the samples. The lysates can be stored at −80° C. until use in the quantification assay.

The PROTEINSIMPLE® SIMON™ western blotting bioassay assay employs a quantitative western blotting immunoassay technique using an antibody specific for filaggrin to quantitatively detect filaggrin in the test samples. Cell samples are lysed and normalized for protein concentration. Normalized samples and molecular weight standards can then be loaded and ran on a denatured protein separation gel using capillary electrophoresis. The proteins in the gel are immobilized and immunoprobed using a primary antibody specific for filaggrin. The immobilized proteins can then be immunoprobed with an enzyme-linked detection antibody that binds the primary antibody. A chemiluminescent substrate solution can then be added to the immobilized proteins to allow chemiluminescent development in proportion to the amount of filaggrin bound in the immobilization. The chemiluminescent development is stopped at a specific time and the intensity of the chemiluminescent signal can be measured and compared to positive and negative controls.

Production of Occludin: Changes in the production of occludin in keratinocytes due to each of the active ingredients, any one of the combination of ingredients, or compositions having said combinations disclosed in the specification can be measured. Occludin is a protein critical to the formulation of tight junctions and the skin's moisture barrier function. A non-limiting example of how occludin production in treated and non-treated keratinocytes can be determined is by the use of a bioassay that analyzes occludin concentration in keratinocyte cell lysates. The bioassay can be performed using PROTEINSIMPLE® SIMON™ western blotting protocol. For the samples, adult human epidermal keratinocytes (HEKa) from Life Technologies (C-005-5C) can be grown at 37° C. and 5% CO2 for 24 hours in EPILIFE™ growth media with calcium from Life Technologies (M-EP-500-CA) supplemented with Keratinocyte Growth Supplement (HKGS) from Life Technologies (S-101-5). HEKa are then incubated in growth medium with test compound/extract, no compound/extract for negative control, or with 1 mM CaCl₂) for positive control for 24 to 48 hours. The HEKa are then washed, collected, and stored on ice or colder until lysed on ice using a lysis buffer and sonication. The protein concentrations of the samples can be determined and used to normalize the samples. The lysates are stored at −80° C. until use in the bioassay.

The PROTEINSIMPLE® SIMON™ western blotting bioassay assay employs a quantitative western blotting immunoassay technique using an antibody specific for occludin to quantitatively detect occludin in the test samples. Cell samples are lysed and normalized for protein concentration. Normalized samples and molecular weight standards are then loaded and ran on a denatured protein separation gel using capillary electrophoresis. The proteins in the gel are then immobilized and immunoprobed using a primary antibody specific for occludin. The immobilized proteins are immunoprobed with an enzyme-linked detection antibody that binds the primary antibody. A chemiluminescent substrate solution is then added to the immobilized proteins to allow chemiluminescent development in proportion to the amount of occludin bound in the immobilization. The chemiluminescent development can be stopped at a specific time and the intensity of the chemiluminescent signal can be measured and compared to positive and negative controls.

Production of Hyaluronic Acid: Changes in the production of hyaluronic acid in human dermal fibroblasts due to each of the active ingredients, any one of the combination of ingredients, or compositions having said combinations disclosed in the specification can be measured. HA is a polysaccharide involved in stabilization of the structure of the matrix and is involved in providing turgor pressure to tissue and cells.

As one non-limiting example, HA production in treated and non-treated adult human dermal fibroblasts (HDFa) cells can be determined using the Hyaluronan DuoSet ELISA kit from R&D Systems (DY3614). In this assay, for production of samples, subconfluent HDFa cells from Cascade Biologics (C-13-5C) are incubated at 37° C. and 10% CO₂ in starvation medium (0.15% fetal bovine serum and 1% Penicillin Streptomycin solution in Dulbecco's Modified Eagle Medium) for 72 hours prior to treatment. The cells are then incubated with fresh starvation medium with either test compound, positive control (phorbol 12-myristate 13-acetate from SIGMA-ALDRICH (P1585) and platelet derived growth factor from SIGMA-ALDRICH (P3201)), or no additive for 24 hours. Media is then collected and frozen at −80° C. until use in the ELISA assay.

Briefly, the ELISA assay employs a quantitative sandwich enzyme immunoassay technique whereby a capture antibody specific for HA can be pre-coated onto a microplate. Standards and media from treated and untreated cells are pipetted into the microplate wells to enable any HA present to be bound by the immobilized antibody. After washing away any unbound substances, an enzyme-linked detection antibody specific for HA is added to the wells. Following a wash to remove any unbound antibody-enzyme reagent, a substrate solution is added to the wells to allow color development in proportion to the amount of HA bound in the initial step. The color development is stopped at a specific time and the intensity of the color at 450 nm can be measured using a microplate reader.

As another non-limiting example, human skin explants can be cultured in survival explants medium at 37° C. in a humidified atmosphere supplemented with 5% CO2. Treatment of the explants can be carried out by topical application of sample product (n=3) on days D0, D2, D3, D6, D8, and D9. The control explants (n=3) receive no treatment except renewal of survival explants medium. Half of the volume of the survival medium can be renewed at days D3, D6, and D8. At D9, three explants of each condition can be taken and cut in half. A half explant is fixed in buffered formalin and the other is frozen at −80° C.

After 48 hours of fixation in ordinary Bouin and 24 hours in formalin, the samples can be dried and soaked in paraffin using an automatic tissue processor Leica TP 1020. Sections of 5 microns can be performed with a microtome (Minot type LEICA RM2125) and mounted on SUPERFROST™ histological slides. Microscopic observations can be performed by optical microscopy, using a LEICA ORTHOPLAN or LEICA DM LB microscope. Images can be taken with an OLYMPUS DP72 camera and CELL\hat{ }D software. General morphology can be examined on paraffin sections stained with Masson's trichrome Goldner variant. The staining of hyaluronic acid can be performed with an anti-hyaluronic acid biotinylated protein (HABP) (SEIKAGAKU ref 400763-1A) diluted to 1/100 for 1 hour at room temperature, with an amplifier system biotin/streptavidin (VECTOR, VECTASTAIN PK-7200).

Inhibition of Hyaluronidase Activity: Changes in the activity of hyaluronidase due to each of the active ingredients, any one of the combination of ingredients, or compositions having said combinations disclosed in the specification can be measured. Hyaluronidase is an enzyme that degrades HA. HA is a polysaccharide involved in stabilization of the structure of the matrix and is involved in providing turgor pressure to tissue and cells. As one non-limiting example, hyaluronidase activity can be determined using an in vitro protocol modified from SIGMA-ALDRICH protocol #EC 3.2.1.35. Briefly, hyaluronidase type 1-S from SIGMA-ALDRICH (H3506) is added to microplate reaction wells containing test compound or controls. Tannic acid can be used as a positive control inhibitor, no test compound can be added for the control enzyme, and wells with test compound or positive control but without hyaluronidase can be used as a background negative control. The wells are incubated at 37° C. for 10 minutes before addition of substrate (HA). Substrate is added and the reactions incubated at 37° C. for 45 minutes. A portion of each reaction solution is then transferred to and gently mixed in a solution of sodium acetate and acetic acid pH 3.75 to stop that portion of the reaction (stopped wells). The stopped wells and the reaction wells should both contain the same volume of solution after addition of the portion of the reaction solution to the stopped wells. Both the reaction wells and the stopped wells are incubated for 10 minutes at room temperature. Absorbance at 600 nm is then measured for both the reaction wells and the stopped wells. Inhibition can be calculated using the following formulas: Inhibitor (or control) activity=(Inhibitor stopped wells absorbance at 600 nm−inhibitor reaction wells absorbance at 600 nm); Initial activity=control enzyme absorbance at 600 nm; Percent Inhibition=[(Initial activity/Inhibitor Activity)*100]−100.

Peroxisome Proliferator-Activated Receptor Gamma (PPAR-γ) Activity: Changes in the activity of PPAR-γ due to each of the active ingredients, any one of the combination of ingredients, or compositions having said combinations disclosed in the specification can be measured. PPAR-γ is a receptor critical for the production of sebum. As one non-limiting example, the activity of PPAR-γ can be determined using a bioassay that analyzes the ability of a test compound or composition to inhibit binding of a ligand. Briefly, fluorescent small-molecule pan-PPAR ligand, FLUORMONET Pan-PPAR Green, available from Life Technologies (PV4894), can be used to determine if test compounds or compositions are able to inhibit binding of the ligand to PPAR-γ. The samples wells include PPAR-γ and fluorescent ligand and either: test compound or composition (test); a reference inhibitor, rosiglitazone (positive control); or no test compound (negative control). The wells are incubated for a set period of time to allow the ligand opportunity to bind the PPAR-γ. The fluorescence polarization of each sample well can then be measured and compared to the negative control well to determine the percentage of inhibition by the test compound or composition.

Cytokine array: Human epidermal keratinocytes are cultured to 70-80% confluency. The media in the plate is aspirated and 0.025% trypsin/EDTA is added. When the cells became rounded, the culture dish is gently tapped to release the cells. The trypsin/EDTA containing cells are removed from the culture dish and neutralized. Cells are centrifuged for 5 min. at 180×g to form a pellet of cells. The supernatant is aspirated. The resulting pellet is resuspended in EPILIFE™ media (Cascade Biologics). The cells are seeded in 6-well plates at approximately 10-20% confluency. After the cells became approximately 80% confluent, the media is aspirated and 1.0 ml of EPILIFE™, along with phorbol 13-Myristate 12-acetate (“PMA”) (a known inducer of inflammation) and the test composition dilutions are added to two replicate wells (e.g., 1.0% (100 μl of 100× stock) and 0.1% (10 μl of 100× stock) test compositions are diluted into a final volume of 1 ml EPILIFE™ Growth Medium). The media is gently swirled to ensure adequate mixing. In addition, 1.0 ml of EPILIFE™ is added to the control wells, with and without additional PMA. The plates are then incubated at 37±1° C. and 5.0±1% CO₂ for approximately 5 hours after dosing. Following this 5-hour incubation, all media is collected in conical tubes and frozen at −70° C.

For analysis, a 16-pad hybridization chamber is attached to 16-pad FAST slides arrayed in triplicate with 16 anti-cytokine antibodies plus experimental controls (WHATMAN BIOSCIENCES), and the slides are placed into a FASTFrame (4 slides per frame) for processing. Arrays are blocked for 15 min. at room temperature using 70 ml S&S PROTEIN ARRAY BLOCKING BUFFER (WHATMAN SCHLEICHER AND SCHEULL). Blocking buffer is removed and 70 ml of each supernatant sample is added to each array. Arrays are incubated for 3 hours at room temperature with gentle agitation. Arrays are washed 3 times with TBS-T. Arrays are treated with 70 ml of an antibody cocktail, containing one biotinylated antibody corresponding to each of the arrayed capture antibodies. Arrays are incubated for 1 hour at room temperature with gentle agitation. Arrays are washed 3 times with TBS-T. Arrays are incubated with 70 ml of a solution containing streptavidin-Cy5 conjugate for 1 hour at room temperature with gentle agitation. Arrays are washed 3 times with TBS-T, quickly rinsed in de-ionized water, and dried.

Slides can be imaged in a PERKIN-ELMER SCANARRAY 4000 confocal fluorescent imaging system. Array images can be saved and analyzed using IMAGING RESEARCH ARRAYVISION software. Briefly, spot intensities are determined by subtracting background signal. Spot replicates from each sample condition can be averaged and then compared to the appropriate controls.

Endothelial Tube Formation: Endothelial tube formation is involved in angiogenesis and micro-vessel capillary formation. Capillary formation and angiogenesis may contribute to redness and rosacea of the skin. The ability for endothelial cells to form tubes in the presence or absence of test extracts and compounds may be determined using a capillary tubule disruption assay with pre-formed primary human umbilical vein endothelial cells (HUVEC) in a cell culture system.

Briefly, HUVECs are cultured in vitro on Extracellular Matrix, which stimulates the attachment and tubular morphogenesis of endothelial cells to form capillary-like lumen structures. These in vitro formed capillary tubules are similar to human blood vessel capillaries in many aspects. The capillary tube assay is based on this phenomenon and is used for evaluation of potential vasculature targeting agents.

HUVEC cultures are grown in a 5% CO₂ 37° C. cell incubator. The full growth medium for HUVECs is Endothelial Cell Basal Medium (EBM) supplemented with 2% fetal bovine serum (FBS), 12 μg/ml bovine brain extract, 1 μg/ml hydrocortisone, and 1 μg/ml GA-1000 (gentamicin-amphothericin). HUVEC cultures between passage 3 and 8 may be used for all assay experiments.

HUVECs are pre-labeled with fluorescent agent Calcein AM and seeded in Extracellular Matrix coated 96-well culture plate with their full growth medium. After about four hours of the morphogenesis process, the endothelial capillary tubes should be formed. Then, test agent in designed doses in 50 μl volume is applied into the formed capillary tubule cultures as treatment conditions. The no-treatment controls can be added with vehicle of test agents. SUTENT®, a FDA approved anti-angiogenic drug one concentration can be included as assay performance control. After about six hours of treatment, the endothelial tubule morphology in each well is examined by microscopy, imaged, and the capillary disrupting activities under treatment conditions can be quantitatively analyzed. Each test conditions can be conducted in duplicate wells, including controls.

Keratinocyte Monolayer Permeability: Changes in the permeability of a keratinocyte monolayer due to each of the active ingredients, any one of the combination of ingredients, or compositions having said combinations disclosed in the specification can be measured. Keratinocyte monolayer permeability is a measure of skin barrier integrity. Keratinocyte monolayer permeability in treated and non-treated keratinocytes can be determined using, as a non-limiting example, the In Vitro Vascular Permeability assay by MILLIPORE (ECM642). This assay analyzes endothelial cell adsorption, transport, and permeability. Briefly, adult human epidermal keratinocytes from Life Technologies (C-005-5C) can be seeded onto a porous collagen-coated membrane within a collection well. The keratinocytes are then incubated for 24 hours at 37° C. and 5% CO₂ in EPILIFE™ growth media with calcium from LIFE TECHNOLOGIES (M-EP-500-CA) supplemented with Keratinocyte Growth Supplement (HKGS) from LIFE TECHNOLOGIES (S-101-5). This incubation time allows the cells to form a monolayer and occlude the membrane pores. The media is then replaced with fresh media with (test sample) or without (non-treated control) test compounds/extracts and the keratinocytes are incubated for an additional 48 hours at 37° C. and 5% CO₂. To determine permeability of the keratinocyte monolayer after incubation with/without the test compound/extract, the media is replaced with fresh media containing a high molecular weight Fluorescein isothiocyanate (FITC)-Dextran and the keratinocytes are incubated for 4 hours at 37° C. and 5% CO₂. During the 4 hours incubation, FITC can pass through the keratinocytes monolayer and porous membrane into the collection well at a rate proportional to the monolayer's permeability. After the 4 hour incubation, cell viability and the content of FITC in the collection wells can be determined. For the FITC content, the media in the collection well is collected and fluorescence of the media determined at 480 nm (Em) when excited at 520 nm. Percent permeability and percent change in comparison to the non-treated controls can be determined by the following equations: Percent Permeability=((Mean Ex/Em of test sample)/Mean Ex/Em untreated control)*100; Percent Change=Percent Permeability of test sample-Percent Permeability of untreated control.

Oil Control Assay: An assay to measure reduction of sebum secretion from sebaceous glands and/or reduction of sebum production from sebaceous glands can be assayed by using standard techniques known to those having ordinary skill in the art. In one instance, the forehead can be used. Each of the active ingredients, any one of the combination of ingredients, or compositions having said combinations disclosed in the specification can be applied to one portion of the forehead once or twice daily for a set period of days (e.g., 1, 2, 3, 4, 5, 6, 7, 8, 9, 10, 11, 12, 13, 14, or more days), while another portion of the forehead is not treated with the composition. After the set period of days expires, then sebum secretion can be assayed by application of fine blotting paper to the treated and untreated forehead skin. This is done by first removing any sebum from the treated and untreated areas with moist and dry cloths. Blotting paper can then be applied to the treated and untreated areas of the forehead, and an elastic band can be placed around the forehead to gently press the blotting paper onto the skin. After 2 hours the blotting papers can be removed, allowed to dry and then transilluminated. Darker blotting paper correlates with more sebum secretion (or lighter blotting paper correlates with reduced sebum secretion.

Erythema Assay: An assay to measure the reduction of skin redness can be evaluated using a MINOLTA chroma meter. Skin erythema may be induced by applying a 0.2% solution of sodium dodecyl sulfate on the forearm of a subject. The area is protected by an occlusive patch for 24 hrs. After 24 hrs, the patch is removed and the irritation-induced redness can be assessed using the a* values of the MINOLTA chroma meter. The a* value measures changes in skin color in the red region. Immediately after reading, the area is treated with the active ingredients, any one of the combination of ingredients, or compositions having said combinations disclosed in the specification. Repeat measurements can be taken at regular intervals to determine the formula's ability to reduce redness and irritation.

Skin Moisture/Hydration Assay: Skin moisture/hydration benefits can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72° C.). Three separate impedance readings can be taken on each side of the face, recorded, and averaged. The T5 setting can be used on the impedance meter which averages the impedance values of every five seconds application to the face. Changes can be reported with statistical variance and significance. Each of the active ingredients, any one of the combination of ingredients, or compositions having said combinations disclosed in the specification can be assayed according to this process.

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether each of the active ingredients, any one of the combination of ingredients, or compositions having said combinations disclosed in the specification is inducing irritation. The measurements can be made on each side of the face and averaged, as left and right facial values. Skin clarity can also be measured using the Minolta Meter. The measurement is a combination of the at, b, and L values of the Minolta Meter and is related to skin brightness, and correlates well with skin smoothness and hydration. Skin reading is taken as above. In one non-limiting aspect, skin clarity can be described as L/C where C is chroma and is defined as (a²+b²)^{1/2}.

Skin Dryness, Surface Fine Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Clinical Grading of Skin Tone Assay: Clinical grading of skin tone can be performed via a ten point analog numerical scale: (10) even skin of uniform, pinkish brown color. No dark, erythremic, or scaly patches upon examination with a hand held magnifying lens. Microtexture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaliness. Skin rough to the touch in some areas; and (1) uneven skin coloration and texture. Numerous areas of scaliness and discoloration, either hypopigmented, erythremic or dark spots. Large areas of uneven color more than 1 cm in diameter. Evaluations were made independently by two clinicians and averaged.

Clinical Grading of Skin Smoothness Assay: Clinical grading of skin smoothness can be analyzed via a ten point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations were made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman et al. (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Skin Firmness Assay with a Hargens Ballistometer: Skin firmness can be measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and are of the replicas covered by wrinkles or fine lines was determined.

Surface Contour of the Skin Assay with a Profilometer/Stylus Method: The surface contour of the skin can be measured by using the Profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Rₐ=Standardize roughness; lₘ=the traverse (scan) length; and y=the absolute value of the location of the profile relative to the mean profile height (x-axis).

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A sunscreen composition comprising avobenzone, octocrylene, octisalate, and aluminum starch octenylsuccinate.

2. The sunscreen composition of claim 1, wherein the composition does not include homosalate.

3. The sunscreen composition of claim 1, wherein the composition includes one or more additional organic UV filters.

4. The sunscreen composition of claim 1, wherein the composition does not include a water-soluble UV filter.

5. The sunscreen composition of claim 1, wherein the composition comprises:
2% to 5% by weight of the avobenzone;
8% to 11% by weight of the octocrylene;
2% to 5% by weight of the octisalate; and/or
0.01% to 5% by weight of the aluminum starch octenylsuccinate.

6. The sunscreen composition of claim 1, further comprising:
water;
an antioxidant;
an emulsifying agent;
a surfactant;
a film-forming agent;
a chelating agent;
a moisturizing agent;
a preservative; and/or
a thickening agent.

7. The sunscreen composition of claim 1, further comprising capryl methicone, ceteareth-25, disodium ethylene dicocamide PEG-15 disulfate (Surfactant), PVP/eicosene copolymer, glyceryl stearate, beeswax, glycerin, disodium EDTA, hydroxyacetophenone, propanediol, xanthan gum, ammonium acryloyldimethyltaurate/VP copolymer, phenoxyethanol, ethylhexylglycerin, dimethicone, and/or silica.

8. The sunscreen composition of claim 7, further comprising niacinamide, encapsulated resveratrol, oligopeptide-1, and/or Opuntia ficus-indica fruit extract.

9. The sunscreen composition of claim 8, further comprising cetearyl alcohol, potassium hydroxide, caprylyl glycol, hexylene glycol, 1,2-hexanediol, decylene glycol, Cestrum latifolium leaf extract, calcium ketogluconate, tripeptide-1, Centella asiatica meristem extract, Silybum marianum extract, 4-t-butylcyclohexanol, sodium PCA, Alpinia galanga leaf extract, and/or Saussurea involucrate extract.

10. The sunscreen composition of claim 1, wherein the composition has a sun protection factor (SPF) of 20 to 50.

11. A method for protecting skin from ultraviolet radiation, the method comprising topically applying to the skin a composition comprising an effective amount of avobenzone, octocrylene, octisalate, and aluminum starch octenylsuccinate.

12. The method of claim 11, wherein the composition does not include homosalate.

13. The method of claim 11, wherein the composition does not include a water-soluble UV filter.

14. The method of claim 11, wherein the composition comprises:
2% to 5% by weight of the avobenzone;
8% to 11% by weight of the octocrylene;
2% to 5% by weight of the octisalate; and/or
0.1% to 3% by weight of the aluminum starch octenylsuccinate.

15. The method of claim 11, wherein the skin is prone to sunburn and/or melanin overproduction after sun exposure and/or application of the composition reduces the risk of sunburn and/or melanin overproduction after sun exposure.

16. The method of claim 11, wherein the skin is not sunburned.

17. The method of claim 11, wherein the skin is treated to reduce or prevent melanin production in the skin.

18. The method of claim 11, wherein the skin is treated to reduce or prevent sunburn.

19. The method of claim 11, wherein oiliness of the skin surface is not significantly increased after application of the composition.

20. The method of claim 11, wherein the composition is combined with one or more other skin care compositions prior to application to the skin.
